(19) United States
(12) Patent Application Publication
(10) Pub. No.: US 2003/0022231 A1
(43) Pub. Date: Jan. 30, 2003

(54) Detection of nucleic acids

(75) Inventors: Lawrence Wangh, Auburndale, MA (US); Kenneth Pierce, Natick, MA (US); Cristina Hartshorn, Needham, MA (US); John Rice, Quincy, MA (US); J. Aquiles Sanchez, Framingham, MA (US)

Correspondence Address: LAHIVE & COCKFIELD, 28 STATE STREET, BOSTON, MA, 02109, US

(73) Assignee: Brandeis University

(21) Appl. No.: 10/242,395

(22) Filed: Sep. 12, 2002

Related U.S. Application Data
(63) Continuation of application No. 09/638,642, filed on Aug. 14, 2000.
(60) Provisional application No. 60/149,013, filed on Aug. 13, 1999.

(51) Int. Cl. C12Q 1/68; C07H 21/04

(52) U.S. Cl. 435/6; 536/24.3

(57) ABSTRACT

Disclosed are compositions, methods, and kits useful for the detection of the presence and/or quantity of one or more chromosomes from single cells, groups of cells, or subcellular compartments. Provided is a lysis buffer for the preparation of substantially accessible nucleic acid molecules from a single cell. Also provided are moderately-repeated highly-conserved nucleic acid sequences, and oligonucleotide primer and probe molecules which hybridize specifically thereto. Methods for the detection of the presence or quantity of one or more chromosomes from a single cell are included, as are methods for the assessment of the reliability of the results of the methods of the invention. Kits for the convenient practice of the invention are also included.

### RELATED APPLICATION

This application claims priority to U.S. Provisional Application No. 60/149,013, filed on Aug. 13, 1999, incorporated herein in its entirety by this reference.

### BACKGROUND OF THE INVENTION

It has become possible to recover and manipulate very small biological samples; in many fields, including diagnostic medicine, forensic science, and biological research, the ability to perform sensitive genetic analyses on these small-scale samples is becoming increasingly necessary. For example, in forensic science, minute tissue or fluid samples may be used to identify the sex or genetic identity of a perpetrator of a crime. Similarly, in diagnostic medicine, minute tumors need to be tested for the presence of cancerous cells, and small samples of amniotic fluid for the presence of genetic abnormalities in a fetus. While such analyses are increasingly possible on a large scale (e.g., by culturing the cells from the sample), production of large-scale samples is in most cases a time-consuming and error-prone process, and in many cases is impossible. Improvements in small-scale genetic analysis have been made, but genetic analyses in samples of only a few cells, a single cell, or even a portion of a cell are still largely hindered by significant technical problems.

Advances in molecular genetics are rapidly generating the tools needed to overcome some of these difficulties, as can be seen in the field of pre-implantation genetic diagnosis (PGD). PGD seeks to identify alleles responsible for many inherited human diseases, and applies this knowledge for the detection of both chromosomal abnormalities and disease-causing alleles in single cells biopsied from human embryos. The resulting information can be used by couples to decide which of their embryos they wish transferred to the uterus to establish a pregnancy. Thus PGD promises an alternative to prenatal diagnosis and abortion of an ongoing pregnancy for couples who want to have a child with certain chromosomes, genes, or genetic alleles, but not other chromosomes, genes, or genetic alleles, e.g., those that result in a child afflicted by a severe disease of genetic etiology. In recent years, several births have been reported following uterine transfer of embryos tested by PGD (Harper, J. C. (1996) J. Assist. Reprod. Genet. 13:90-95; Verlinsky, Y. and Kuliev, A. (1998) J. Assist. Reprod Genet. 15:215-218). Despite these favorable outcomes, PGD remains a difficult and only moderately reliable technology and is currently utilized on an experimental basis at select IVF clinics around the world.

At least 155 inherited diseases are due to genes on the X-chromosome and are therefore expressed in 50% of the sons born to mothers who carry one abnormal allele (McKusick, V. A. (1998) Catalogs of Human Genes and Genetic Disorders. Johns Hopkins Univ. Press: Baltimore). Several diseases related to infertility are linked to the Y chromosome (Lahn, B. T., and Page, D. C. (1997) Science 278:675-680). PGD by means of fluorescent in situ hybridization (FISH) with probes to the X and Y chromosomes has been used in several cases involving X-linked diseases (Griffin, D. K. et al. (1994) J. Assist. Reprod. Genet. 11:132-143) and also has the potential advantage of detecting sex chromosome aneuploidies. The efficiency and accuracy of this technique, however, is highly dependent on technical skill and experience. Poor fixation and lack of stringent scoring criteria can reduce the reliability of FISH (Munne, S. et al. (1998) Mol. Hum. Reprod. 4:863-870). In addition, FISH cannot be extended to distinguish allelic variants of single copy genes.

An alternative to FISH makes use of the polymerase chain reaction (PCR) to amplify one or more DNA sequences in a single cell. Conventional PCR involves repeated cycles of sequence amplification that are typically continued until accumulation of all amplicons in the reaction stops. Amplification is then followed by some form of analysis, such as gel electrophoresis. Conventional PCR is only semi-quantitative at best and reveals little about the kinetics of amplicon accumulation.

PCR was the first method used to identify the sex of embryos for couples known to be at risk for transmitting X-linked diseases (Handyside, A. H. et al. (1990) Nature 344:768-770). The test involved amplification of a highly reiterated sequence of the Y chromosome in single biopsied cells. Only embryos that did not generate the Y-specific product were identified as female and transferred to the uterus, thereby avoiding the birth of potentially afflicted males. Several pregnancies were established using female embryos. The early cases also illustrated some of the risks inherent to PCR-dependent PGD, particularly misdiagnosis and transfer of male embryos if PCR fails and no sequences, including those of the Y-chromosome, are amplified (Hardy, K. and Handyside, A. H. (1992) Arch. Pathol. Lab. Med. 116:388-92). Subsequent protocols reduced this risk by coamplification of reiterated sequences of the X-chromosome which served as an internal control for cell lysis and overall PCR (Kontogianni, E. H. et al. (1991) Co-amplification of X- and Y-Specific sequences for sexing pre-implantation human embryos. In: Verlinsky, Y. and Kuliev, A. (eds.) Preimplantation Genetics. Plenum Press: New York, pp. 139-145; Kontogianni, E. H. et al. (1996) J. Assist. Reprod. Genet. 13:125-132); Strom, C. M. et al. (1991) J. In vitro Fert. Embryo Transf. 8:225-9; and Grifo, J. A. et al. (1992) J. Amer. Med. Assoc. 268:727-729). Nevertheless, high rates of misdiagnosis (3-8%) and amplification failures (7-20%) in those reports lead most investigators to abandon this approach in favor of other PCR strategies or FISH.

In an effort to increase the specificity of PCR, investigators turned to amplification of single copy genes of the Y-chromosome. In order to achieve the required sensitivity when starting with single cells, nested PCR was utilized. Using this approach, male and female blastomeres have been distinguished via PCR amplification of the SRY gene (Cui, K. H. et al. (1994) Lancet 343:79-82), the amelogenin gene (Levinson, G. et al. (1992) Hum. Reprod. 7:1304-1313), and the ZFY gene (Chong, S. S. et al. (1993) Hum. Mol. Genet. 2:1187-1191). Homologous but non-identical copies of both the amelogenin gene and ZFY gene are also located on the X-chromosome and can serve as internal controls for successful amplification, using the same sets of primers. Y-chromosome specific sequences are distinguished from their X-chromosome homologues using gel electrophoresis. Nevertheless, the nested PCR strategy requires more cycles of amplification, increased sample handling, and some method for distinguishing the PCR products. These additional steps increase the time required to complete the assay and the risk of contaminating either the sample or the laboratory.

PCR analysis of single copy genes also is plagued by the problem of “allele drop-out”, the selective failure to amplify one of the target sequences present in the starting cell (reviewed in Lissens, W. and Sermon, K. (1997) Hum. Reprod. 12:17561761). Improved protocols for cell lysis and DNA denaturation prior to PCR have decreased rates of allele drop-out (Gitlin, S. A. et al. (1996) J. Assist. Reprod. Genet. 13:107-111; Ray, P. F. et al. (1996) J. Assist. Reprod. Genet. 13:104-106; El-Hashemite, N. and Delhanty, J. D. A. (1997) Mol. Hum. Reprod. 3:975-958), as has the use of fluorescently-labeled primers (Findlay, I. et al. (1995) Hum. Reprod. 10:1609-1618), but this phenomenon continues to be a problem.

### SUMMARY OF THE INVENTION

The invention pertains to improved compositions and methods for the detection and/or quantification of specific nucleic acid sequences (e.g., sequences within chromosomes) in groups of cells (e.g., fewer than 10 cells, 5 or fewer cells, or 2 or fewer cells), single cells, or parts of cells (e.g., organelles), such as that required for preimplantation genetic diagnosis (PGD), prenatal diagnosis, or forensic science. The invention employs an amplification (e.g., real-time PCR) technique in which a moderately-repeated highly-conserved sequence of a target nucleic acid molecule is amplified by means of specific oligonucleotide primers, and the amplified product (amplicon) is detected in real time by a labeled oligonucleotide probe included in the amplification reaction. Use of a moderately-repeated highly-conserved sequence eliminates the need for nested PCR and makes it possible to amplify and detect the plurality of copies of the nucleic acid sequence with a single set of primers and a single labeled probe. The moderately-repeated sequence also circumvents the problem of allele drop-out, since the results are unaffected even if several of the target copies are not amplified. Using the methods of the invention, cell lysis, gene amplification, and realtime analysis of samples is simple and convenient, and can be completed in a few hours. The methods of the invention provide highly sensitive and accurate (e.g. 99.6%) detection of a specific nucleic acid molecule (e.g., a chromosome) from virtually any type of single cell, group of cells, or part of a cell (e.g., an organelle).

The invention may also be practiced utilizing oligonucleotide primer molecules which are detectably labeled such that the label is detectable only when the primer is in a hybridized state or only when the primer is in an unhybridized state. In this situation, the labeled oligonucleotide probe may be omitted from the reaction.

The methods of the invention may also be used to detect and/or quantify a desired nucleic acid molecules in a cell, a group of cells (e.g., fewer than 10 cells, 5 or fewer cells, or 2 or fewer cells), or a part of a cell through the amplification and detection of a single-copy gene that is specific to the desired nucleic acid molecule. Although the use of a single-copy gene is prone to the problem of allele drop-out, as discussed above, the compositions and methods of the invention not only render the nucleic acid molecules in the sample more accessible to oligonucleotide primer and probe molecules in the reaction (thus decreasing the likelihood of a skipped amplification initiation event), but also increase the sensitivity of detection of the desired nucleic acid molecules through decreased sample contamination and loss, such that amplification of a single-copy gene as a means for reliably detecting the presence and/or quantity of a selected nucleic acid molecule with which the single-copy gene is associated is possible.

In one aspect, the probe is labeled such that the molecule is detectable by an increase or decrease in the detectable signal in the hybridized or unhybridized state, and a hybridization event may be detected without further addition to or modification of the sample. In this circumstance, the amplification reactions of the invention are carried out in sealed tubes or other suitable containers, and do not require the use of nested-PCR primers or electrophoretic analysis of the resulting amplicons, greatly reducing the risk of contamination within the laboratory.

In one embodiment, the present invention provides a protease-based lysis buffer for the preparation of substantially accessible nucleic acids from a cell, portions of a cell, or from groups of cells, containing an ionic detergent, a protease, and a buffering agent. In a preferred embodiment, the protease is proteinase K. In a particularly preferred embodiment, the temperature of the buffer is about 50° C., the protease is proteinase K, the ionic detergent is sodium dodecyl sulfate, and the buffering agent is about 0.5 mM-100 mM Tris HCl, pH 7.53 at 50° C., with 5 mM Tris HCl, pH 7.53 at 50° C. being most preferred.

In another embodiment, the invention provides an alkaline lysis buffer that does not contain DTT or other reducing agents, for use in the methods of the invention. The alkaline lysis buffer without DTT may be used in the methods of the invention instead of the protease-based lysis buffer.

In another embodiment, the invention provides a method for preparing a nucleic acid sample from a cell for an amplification reaction. In this process, a protease-based lysis buffer containing an ionic detergent, a protease, and a buffering agent is added to the cell to form a mixture. This mixture is incubated at a temperature at which the protease is active, such that substantially accessible nucleic acids are obtained.

In another embodiment, the present invention provides an isolated nucleotide molecule having a sequence that is:

a) repeated greater than 3-100 times within the genome of a cell, and

b) sufficiently conserved such that the plurality of the repeats of the sequence are able to hybridize to at least two non-overlapping nucleotide primers, wherein these primers may be utilized to amplify the repeated sequence.

In another embodiment, the present invention provides a primer comprised of a linear sequence of typically about 6-50 nucleotides that is sufficiently complementary to a moderately-repeated highly-conserved nucleic acid sequence in the genetic complement of a cell to permit hybridization of the linear sequence to a plurality of the copies of the moderately-repeated highly-conserved nucleic acid sequence in the genetic complement of the cell. In one embodiment, the cell is a mammalian cell. In a preferred embodiment, the cell is a human cell.

In another embodiment, the present invention provides a probe comprised of a linear sequence of typically about 6-50 nucleotides that is sufficiently complementary to a moderately-repeated highly-conserved nucleic acid sequence in the genetic complement of a cell to permit hybridization of the linear sequence to a plurality of the copies of the moderately-repeated highly-conserved nucleic acid sequence in the genetic complement of the cell. In one embodiment, the cell is a mammalian cell. In a preferred embodiment, the cell is a human cell.

In another embodiment, the present invention provides a nucleic acid primer specific for human chromosome 17, wherein the primer is sufficiently complementary to a moderately-repeated highly-conserved sequence contained within human chromosome 17 to permit hybridization to a plurality of the copies of this moderately-repeated highly-conserved sequence within human chromosome 17.

In another embodiment, the present invention provides a nucleic acid primer specific for the human Y chromosome, wherein the primer is sufficiently complementary to a moderately-repeated highly-conserved sequence contained within the human Y chromosome to permit hybridization to a plurality of the copies of this moderately-repeated highly-conserved sequence within the human Y chromosome.

In another embodiment, the invention provides methods for selecting pairs of best-possible primers, wherein the best-possible primers are those that optimize a number of parameters in an amplification reaction, as compared to other pairs of primers. Best-possible primers are also those that minimize the production of nonspecific amplicons in an amplification reaction, as compared to other pairs of primers.

In another embodiment, the invention provides methods for producing gene-deleted DNA, wherein a specific sequence within a nucleic acid sample is prevented from amplifying or replicating in an in vitro reaction.

The invention also provides, in another embodiment, a method of detecting the presence or quantity of one or more selected nucleic acid molecules (e.g., a chromosome) or portions thereof in a nucleic acid sample. In this method, the nucleic acid sample is contacted with at least two nucleic acid primers sufficiently complementary to a target moderately-repeated highly-conserved nucleic acid sequence found within the selected nucleic acid molecule or portion thereof such that they are able to hybridize with a plurality of the copies of the target moderately-repeated highly-conserved sequence present in the sample. This targeted moderately-repeated highly-conserved nucleic acid sequence is amplified by an amplification reaction, and the amplified moderately-repeated highly-conserved nucleic acid sequence is detected as indicative of the presence or quantity of the selected nucleic acid molecule (e.g., chromosome) or portion thereof.

In another embodiment, the invention provides a method of detecting the presence or quantity of a nucleic acid molecule (e.g., a chromosome) or portion thereof in a nucleic acid sample, in which the sample is contacted with at least two nucleic acid primers, at least one of which is detectably labeled, and each of which is sufficiently complementary to a moderately-repeated highly-conserved nucleic acid sequence found within the nucleic acid molecule (e.g., a chromosome) or portion thereof that it is able to specifically hybridize to the plurality of the copies of this moderately-repeated highly-conserved nucleic acid sequence. The moderately-repeated highly-conserved nucleic acid sequence is amplified, and the amplified nucleic acid sequence is detected at selected times of amplification (e.g., cycles) by measuring the label associated with the primer hybridized to the nucleic acid sequence. The quantity of the amplified nucleic acid sequence at a first selected time (e.g., cycle) of amplification and the quantity of this amplified sequence at a later second selected time (e.g., cycle) of amplification can be compared to predetermined quantity values for the first and second selected times as an indication of the efficiency or accuracy of the amplification reaction. These quantities are utilized as an indication of the presence or quantity of the nucleic acid molecule (e.g., a chromosome) or portion thereof in the nucleic acid sample.

In another embodiment, the invention provides a method of detecting the presence or quantity of a nucleic acid molecule (e.g., a chromosome) or portion thereof in a nucleic acid sample. In this method, the sample is contacted with at least two nucleic acid primers, each primer being sufficiently complementary to a target moderately-repeated highly-conserved nucleic acid sequence found within the nucleic acid molecule (e.g., a chromosome) or portion thereof such that they are able to hybridize with a plurality of these sequences. The sample is also contacted with at least one detectably labeled probe which is sufficiently complementary to the above-mentioned moderately-repeated highly-conserved nucleic acid sequence such that it hybridizes to a plurality of the copies of the sequence present in the sample. The moderately-repeated highly-conserved nucleic acid sequence is amplified by an amplification reaction, and the amplified moderately-repeated highly-conserved nucleic acid sequence is detected at selected times (e.g., cycles) of amplification by measuring the label associated with the probe either hybridized or not hybridized to the target moderately-repeated highly-conserved sequence. The quantity of amplified moderately-repeated highly-conserved nucleic acid sequence at a first selected time (e.g., cycle) of amplification and the quantity of amplified moderately-repeated highly-conserved nucleic acid sequence at a later second selected time can be compared to predetermined quantity values for the first and second times of amplification as an indication of the efficiency or accuracy of the amplification reaction; and these values are utilized as an indication of the presence or quantity of the selected nucleic acid molecule (e.g., the chromosome) or portion thereof.

In another embodiment, the invention provides a method for detecting and/or quantifying a nucleic acid of interest from a single cell, 2 or fewer cells, 5 or fewer cells, or fewer than 10 cells. In this method, a protease-based lysis buffer containing an ionic detergent, a protease, and a buffering agent is added to the cell to form a mixture. This mixture is incubated at a temperature at which the protease is active, for a period of time such that proteins in the sample are substantially degraded, the protease is inactivated, and an amplification reagent which amplifies the specific nucleic acid molecule is added to the mixture.

The invention further provides, in another embodiment, a method for the detection and/or quantification of a nucleic acid of interest from a single cell, 2 or fewer cells, 5 or fewer cells, or fewer than 10 cells in one reaction vessel. In this process, a protease-based lysis buffer containing a protease, an ionic detergent, and a buffering agent is added to the cell in a reaction vessel to form a mixture. The mixture is incubated at a temperature at which the protease is active, and an amplification reagent which amplifies the specific nucleic acid molecule of interest is added to the vessel. In a further embodiment, the method is performed in a single sealed reaction vessel, wherein the amplification reagent is added to the lysis buffer, and wherein polymerase and magnesium molecules are added in a form such that they are made available for the amplification step only after the protease is inactivated. In a preferred embodiment, the polymerase and magnesium molecules are encased in wax.

In another embodiment, the invention provides a kit for detecting the presence or quantity of a nucleic acid molecule (e.g., a chromosome) in a cell or group of cells, containing a protease-based lysis buffer comprising an ionic detergent, a protease, and a buffering agent, and at least two oligonucleotide primer molecules which specifically hybridize to a moderately-repeated highly-conserved sequence of the nucleic acid molecule (e.g., the chromosome) to be detected. In a preferred embodiment, at least one of the oligonucleotide primers is detectably labeled such that the label is detectable only when the primer is hybridized to the sequence to which it is complementary. In another preferred embodiment, the kit further includes a detectably labeled oligonucleotide probe which specifically hybridizes to a plurality of the copies of the moderately-repeated highly-conserved sequence of the nucleic acid molecule (e.g., the chromosome) to be detected. In a particularly preferred embodiment, the label of the oligonucleotide probe is detectable only when the probe is hybridized to the sequence to which it is complementary. In another embodiment, the kit further contains instructional materials.

In another embodiment, the invention provides a kit for detecting the presence or quantity of the human Y chromosome in a human cell, containing a protease-based lysis buffer comprising an ionic detergent, a protease, and a buffering agent, and at least two oligonucleotide primer molecules which specifically hybridize to a moderately-repeated highly-conserved sequence of the human Y chromosome. In a preferred embodiment, at least one of the oligonucleotide primers is detectably labeled such that the label is detectable only when the primer is hybridized to the sequence to which it is complementary. In another preferred embodiment, the kit further includes a detectably labeled oligonucleotide probe which specifically hybridizes to a plurality of the copies of the moderately-repeated highly-conserved sequence of the Y chromosome to be detected. In a particularly preferred embodiment, the label of the oligonucleotide probe is detectable only when the probe is hybridized to the sequence to which it is complementary.

In another embodiment, the invention provides a kit for detecting the presence or quantity of human chromosome 17 in a human cell, containing a protease-based lysis buffer comprising an ionic detergent, a protease, and a buffering agent, and at least two oligonucleotide primer molecules which specifically hybridize to a moderately-repeated highly-conserved sequence of human chromosome 17. In a preferred embodiment, at least one of the oligonucleotide primers is detectably labeled such that the label is detectable only when the primer is hybridized to the sequence to which it is complementary. In another preferred embodiment, the kit further includes a detectably labeled oligonucleotide probe which specifically hybridizes to a plurality of the copies of the moderately-repeated highly-conserved sequence of human chromosome 17 to be detected. In a particularly preferred embodiment, the label of the oligonucleotide probe is detectable only when the probe is hybridized to the sequence to which it is complementary.

### BRIEF DESCRIPTION OF THE DRAWINGS

FIG. 1: Graphical representation of a real time polymerase chain reaction utilizing molecular beacon technology (MB-PCR). The cycle profile of the preferred amplification reaction utilized in the methods of the invention is shown in Panel A, and the corresponding conformational changes that occur in the molecular beacon-tagged oligonucleotide and in the target DNA during the amplification reaction are shown in Panel B. Filled circles represent the quenching moiety on the 3′ end of the beacon, while open circles represent the fluorescent moiety, (which may or may nor fluoresce) on the 5′ end of the beacon. Panel C shows an amplification plot of fluorescence readings of U2 genes in individual female lymphocytes after background is subtracted. The detection threshold is shown as a dotted line. The threshold cycle (C_{T}) for each sample is determined by the point at which the fluorescence plot crosses this line. Final fluorescence is measured at cycle 38.

FIG. 2: Scatter diagrams of threshold cycle (C_{T}) and final fluorescence values for an initial series of lymphocyte samples. Panels A and B show TSPY and U2 signals, respectively, from male lymphocytes. Panels C and D show TSPY and U2 signals, respectively, from female lymphocytes. Panels E and F show TSPY and U2 signals, respectively, from no-cell controls. Final fluorescence was measured at cycle 38.

FIG. 3: Scatter diagrams of threshold cycle (C_{T}) and final fluorescence values from blastomeres and control lymphocytes assayed in parallel. Panels A and B show TSPY and U2 signals, respectively, from male lymphocytes. Panels C and D show TSPY and U2 signals, respectively, from female lymphocytes. Panels E and F show TSPY and U2 signals, respectively, from blastomeres generating both signals. Panels G and H show TSPY and U2 signals, respectively, from blastomeres generating only one of those signals. All no-cell controls lacked signals and are not depicted. Robust signals used for gender diagnosis are those within the area bounded by the broken lines. PCR conditions and molecular beacon probe preparations differ from those used for samples shown in FIG. 2. Final fluorescence was measured at cycle 38.

FIG. 4: Table depicting the mean threshold cycle (C_{T}) and cycle 38 fluorescence values for TSPY and U2 in two experimental series using lymphocytes and blastomeres.

FIG. 5: Table depicting the evaluation of real-time PCR for gender diagnosis of lymphocytes and blastomeres.

FIG. 6: Table depicting the diagnostic concordance among blastomeres from the same embryo.

FIG. 7: Plot of cycle threshold (C_{T}) values for comparison of protease-based lysis buffer, heat denaturation in water, and freeze-thaw in water lysis methods.

FIG. 8: Plot of cycle threshold (C_{T}) values for comparison of alkaline lysis with and without different concentrations of DTT.

FIG. 9: Plot of cycle threshold (C_{T}) values for comparison of protease-based lysis buffer and alkaline lysis without DTT.

FIG. 10: Plot of cycle threshold (C_{T}) values for comparison of different detergents in the protease-based lysis buffer.

FIG. 11: Plot of cycle threshold (C_{T}) values for comparison of protease-based lysis buffer with and without magnesium chloride.

FIG. 12: Graph comparing protease-based lysis buffer with commercially available lysis buffers.

### DETAILED DESCRIPTION OF THE INVENTION

Definitions

The term “nucleic acid molecule” includes DNA molecules (e.g., cDNA or genomic DNA), RNA molecules (e.g., mRNA) and analogs of the DNA or RNA generated using nucleotide analogs. The nucleic acid molecule can be single-stranded or double-stranded, but preferably is double-stranded DNA. For the purposes of the invention, it is understood that nucleic acid molecules having modified base structures (e.g., a synthetic peptidic backbone, as is the case with peptide nucleic acid molecules) are also intended to be encompassed by this term. An “accessible” nucleic acid molecule includes nucleic acid molecules which may readily hybridize with oligonucleotide primer and/or probe molecules, and which may be readily replicated (e.g., a nucleic acid molecule which is substantially free of bound protein molecules).

The term “isolated nucleic acid molecule” includes nucleic acid molecules that are separated from nucleoprotein structures that comprise the natural source of the nucleic acid. For example, with respect to genomic DNA, the term “isolated” includes nucleic acid molecules that are separated from the chromatin, chromosome, or chromosomes or other naturally occurring structures in a cell, cell nucleus, or other subcellular organelle or particle. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

The term “moderately-repeated highly-conserved nucleic acid sequence” includes a nucleic acid sequence found in approximately 3-100 copies within a specific selected nucleic acid molecule (e.g., a chromosome) and sufficiently conserved such that the plurality of the copies of the sequence are able to hybridize to at least two distinct oligonucleotide primer molecules, such that the sequence may be amplified under stringent hybridization conditions by said primer molecules.

The terms “hybridize” or “hybridization” are art-known and include the hydrogen bonding of complementary DNA and/or RNA sequences to form a duplex molecule. As used herein, hybridization takes place under conditions that can be adjusted to a level of stringency that prevents base-pairing between a first oligonucleotide primer or oligonucleotide probe and a target sequence if the complementary sequences are mismatched by as little as one base-pair. Thus, the term “stringent conditions” for hybridization includes conditions that prevent base-pairing between a first oligonucleotide primer or oligonucleotide probe and a target sequence if the complementary sequences are mismatched by at least one base-pair.

The term “genome” includes the entirety of the genetic information contained in the chromosome(s) of a cell or a subcellular organelle.

The terms “complete genome DNA” and “CG-DNA” include the full genetic complement of an organism, cell, organelle, part of a cell, or virus (whether prepared as substantially purified DNA, chromatin, or a nucleus) that is available to replicate or amplify in either a living cell or an in vitro reaction (e.g., the polymerase chain reaction).

The terms “gene-deleted DNA” and “GD-DNA” include the genetic complement of an organism, cell, organelle, part of a cell, or virus (whether prepared as substantially purified DNA, chromatin, or a nucleus) that is naturally occurring or chemically and/or enzymatically treated in vitro in a manner that selectively compromises or limits the capacity of one or more specific sequences within that genome to replicate or amplify an in vitro reaction (e.g., the polymerase chain reaction).

The terms “sequence specific replication inhibitor” and “SSRI” include any drug, chemical, macromolecule, nucleic acid (natural or synthetic), biochemical, enzyme, agent, or compound that can be used to prevent, block, inhibit, delay, or otherwise impede the first replication or repeated replication events of a selected DNA sequence within a genome. An SSRI may also inhibit secondary amplification of the same sequence that would otherwise accumulate in an amplification reaction. However, in order to be an SSRI, the compound need not inhibit secondary amplification of a specific amplicon. In contrast to the selected DNA sequence, all, or substantially all, other sequences within the genome are or can be amplified or replicated under the conditions in which amplification or replication of the selected sequence is prevented by an SSRI.

The term “genetic complement of a cell” includes all genetic material in a cell. This genetic material may include not only the standard chromosomal complement of a cell, but also epigenetic nucleic acid material, such as plasmid(s) or viral nucleic acid sequences which have been incorporated into the chromosome(s) of the cell. This term includes nucleic acid sequences which are present in the cell but which did not originate in the cell.

The term “chromosome” includes a nucleic acid molecule carrying a number of genes which forms the structural unit of genetic material in the genome of a cell. Eukaryotic cells typically have multiple different chromosomes that are generally located in the nucleus of the cell and certain subcellular organelles (e.g., mitochondria), while prokaryotic cells typically have only one circular chromosome located in the cytoplasm of the cell.

The term “cell” includes prokaryotic or eukaryotic cells, and includes eubacterial, bacterial, fungal, plant, insect, animal, and human cells or groups of cells having one or more of these different cell types. The term “part of a cell” includes subcellular compartments such as organelles (e.g., chloroplasts, nuclei, or mitochondria) or combinations of organelles.

The terms “nucleic acid primer”, “primer molecule”, “primer”, and “oligonucleotide primer” include short (between about 10 and about 75 bases) single-stranded oligonucleotides which, upon hybridization with a corresponding template nucleic acid molecule, serve as a starting point for synthesis of the complementary nucleic acid strand by an appropriate polymerase molecule. Primer molecules may be complementary to either the sense or the anti-sense strand of a template nucleic acid molecule.

The terms “best-possible primers” and “BP primers” include one or more pairs of primers for a specific amplicon that generate the fewest non-specific amplicons in an amplification reaction carried out under conditions that are permissive for non-specific amplicon formation. BP primers can be identified by one or both of the following methods: 1) by measuring the reliability of specific amplicon amplification in a reaction (e.g., a PCR reaction) initiated with fewer than 10, 5 or fewer, 2 or fewer, or only one copies of the target sequence; in such samples, BP primers generate specific amplicons most reliably and with the least amount of quantitative variation; 2) by determining which primers (among different tested sets) result in minimal non-specific amplicon amplification in reactions initiated with GD-DNA and maximal specific amplicon amplification in reactions initiated with CG-DNA.

The terms “amplification” or “amplify” include the reactions necessary to increase the number of copies of a nucleic acid sequence (e.g., a DNA sequence). For the purposes of this invention, amplification refers to the in vitro exponential increase in copy number of a target nucleic acid sequence, such as that mediated by the polymerase chain reaction. However, any amplification reaction may be efficaciously employed, such as rtPCR (the experimental embodiment set forth in Mullis (1987) U.S. Pat. No. 4,683,202), the ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), the transcriptional amplification system (Kwoh et al. (1989) Proc. Natl. Acad. Sci. USA 86:1173-1177), Q-Beta Replicase (Lizardi et al. (1988) Bio/Technology 6:1197), and rolling circle replication (Lizardi et al. U.S. Pat. No. 5,854,033; and Lizardi et al. (1998) Nat. Genet. 19: 225-232).

The term “amplicon” includes the target amplified nucleic acid sequence.

The term “specific amplicon” includes a DNA sequence amplified in an amplification reaction that constitutes the intended or expected sequence to be amplified in said reaction. Typically, a specific amplicon is a unique sequence or comprises a small set of sequences with known or expected length. Typically, a specific amplicon is generated using one or more sets of primers of known sequence and hybridization characteristics.

The term “non-specific amplicon” includes one ore more DNA sequences, often of unknown composition, amplified in an amplification reaction from sites within a genome that are either not known or intended as template sites for a known set of primers.

The terms “cycle threshold” and “C_{T}” include a point during an amplification reaction when amplicon accumulation is first detected. Amplicon accumulation is first detected when the fluorescence of a hybridized probe molecule exceeds a threshold value set at approximately 10 standard deviations above background. The C_{T} value reflects both the number of copies of the target sequence available at the start of the reaction and the overall rate of target amplification.

The term “efficiency of amplification” includes the rate at which new amplicons are generated during an amplification reaction. This is measured as a ratio of the relative amount of amplicons at a first time point in an amplification reaction (e.g., a first cycle of a cyclic amplification reaction) to that at a second later time point in the same amplification reaction (e.g., a second later cycle of a cyclic amplification reaction). It is understood that the rate of amplification may change within any given amplification reaction from the earlier time points (e.g., cycles of amplification) to the later ones as a function of available reagents. Thus, any comparison of the efficiency of an amplification reaction (e.g., to a standard or control reaction) must be made using a ratio of the amplicons present at the same two time points during the amplification reaction.

The term “robust reaction” includes a reaction in which said reaction yields a C_{T} value not greater than 3 standard deviations above the mean and final fluorescence not less than 3 standard deviations below the mean.

The term “robust signal” includes the fluorescence signal measured from a robust reaction.

The term “diagnostic accuracy” includes the percentage of samples correctly scored for the presence or absence of a target sequence based on a robust signal.

The term “diagnostic utility” includes the percentage of samples that generate any detectable fluorescence signal.

The term “diagnostic efficiency” includes the percentage of samples in which the detected signals are strong enough to be scored as robust signals.

The terms “nucleic acid probe”, “probe molecule”, and “oligonucleotide probe” include defined nucleic acid sequences complementary to a target nucleic acid sequence to be detected such that the probe will hybridize to the target. Probes are typically detectably labeled, such that the hybridization of the probe to the target sequence may be readily assessed.

The term “detectable label” includes moieties which provide a signal which may be readily detected and, in some embodiments, quantitated. Such labels are well-known to those in the art and include chemiluminescent, radioactive, fluorescent, or colored moieties, or enzymatic groups which, upon incubation with an appropriate substrate, provide a chemiluminescent, fluorescent, radioactive, or calorimetric signal. Methods of detection of such signals are also well-known in the art.

The term “fluor” includes a molecule which absorbs light energy at a selected first wavelength and which emits light energy (fluoresces) at a selected second wavelength. The term “quencher” includes a molecule which prevents the emission of light energy from a fluor, either by absorbing all of the emitted energy itself, by absorbing the light energy which would enable the fluor to fluoresce before it is able to contact the fluor, or by noncovalently joining with the fluor in a manner that renders the joint compound nonfluorescent. The spatial relationship between the fluor and the quencher determines the degree to which the fluorescence of the fluor is quenched—the closer the physical proximity of the molecules, the greater the quenching effect.

The term “lysis buffer” includes reagents which function to rupture a cell, generally through disruption of the cellular membrane and destruction or denaturation of its protein components, thereby rendering the nucleic acid components of the cell available to subsequent biochemical manipulation or analysis. Such lysis buffers may also include components (e.g., buffers) which function to stabilize the desired cellular nucleic acids.

The term “ionic detergent” includes detergent molecules having a charge, either negative (an anionic detergent) or positive (a cationic detergent). Examples of such ionic detergents are described herein, and include sodium dodecyl sulfate (SDS) and lithium lauryl sulfate (LLS).

The term “protease” includes molecules that degrade one or more target proteins. Typically, proteases are specific for a particular three-dimensional protein conformation or amino acid sequence, and can degrade any protein having that preferred conformation or sequence. Proteases have defined environmental conditions (e.g., temperature, salt concentration, and pH) for optimal function. Such conditions and the methods by which these conditions may be ascertained are well known to those skilled in the art.

The term “buffering agent” or “buffer” includes compounds that act to maintain the pH of a solution by maintaining the relative levels of hydrogen and hydroxyl ions in the solution. Buffers have specific pH ranges at which they are functional, and their function is frequently temperature-dependent. Buffers and the temperature-dependence of the buffering capacity thereof are well known to those skilled in the art.

The term “reaction vessel” includes any three-dimensional containment unit of a scale appropriate to the volume of the reaction and of a material commensurate with the conditions, components, and detectable labels of the reaction (e.g., one which can withstand the temperature at which the incubation will take place, or one which is optically clear in order to readily permit the detection of emitted light). Non-limiting examples of reaction vessels include microfuge tubes, slides, matrices, and the like.

The term “real time”, with respect to an amplification reaction, refers to the method by which the amplification reaction is analyzed. For example, in a “real-time” amplification reaction, accumulation of amplicon or product is measured during the progression of the reaction, as opposed to after the reaction is complete.

I. Cellular Lysis and Preparation of Nucleic Acid Samples

A significant barrier to the detection of a desired nucleic acid molecule (e.g., a chromosome) from a cell or a part of a cell (e.g., an organelle) through amplification techniques is the reliability of initiation of the amplification reaction. The preparation of nucleic acid molecules from whole cells typically requires cell lysis, nucleic acid extraction, and nucleic acid purification steps. When working with a large sample size, the loss incurred at each of these steps may be tolerated; however, when working with a single cell or a part of a cell (e.g., an organelle), any loss of genetic information may result in the loss of the target sequence altogether from the amplification reaction, thereby unacceptably skewing the results. Second, the prepared nucleic acids need to be available for hybridization to amplification primers. In the cell, nucleic acid molecules are typically associated with proteins—either transcription factors, nucleases, enzymes, or packaging proteins. The presence of any such protein at the site of primer binding may prevent hybridization or extension of the nucleic acid strand being synthesized, both of which may result in a skipped amplification initiation event. Since the amplicon product during amplification is geometrically or exponentially increased in number, early skipped rounds will significantly decrease the product produced at the end, possibly giving no signal or reduced signal, and rendering accurate quantitative analysis of the target nucleic acid molecule in the sample difficult.

The invention provides a method for preparing the total nucleic acid content of the cell in a single reaction vessel without further purification or manipulation steps. In this method, the cell is lysed by the addition of a lysis buffer also provided by the invention. The cellular proteins associated with the nucleic acid that might interfere with the amplification of many sequences within the genome are degraded, without significant DNA degradation, in order to render substantially all possible sequences within the genome accessible for subsequent amplification.

Protease-Based Lysis Buffer

The protease-based lysis buffer of the invention is composed of an ionic detergent, a protease, and a buffering agent. The preferred protease-based lysis buffer of the invention is composed of sodium dodecyl sulfate, proteinase K, and Tris, pH 8.3 at 25° C. For convenience, the buffer may be prepared in advance at room temperature and stored at between about 0° C. and about −20° C. Each of these components is described further below.

1. Detergent

The detergent is an essential component of the protease-based lysis buffer of the invention, and serves multiple functions necessary for the preparation of cellular nucleic acids. Primary among these functions is the activity of the detergent to disrupt the cellular membrane. There are several types of detergents commonly available. These include ionic, non-ionic, and amphoteric detergents. Ionic detergents are detergent species bearing a net charge, either negative (anionic detergents) or positive (cationic detergents). Examples of anionic detergents include alkyl aryl sulphonates (e.g., dodecyl benzene), long chain (fatty) alcohol sulphates, olefine sulphates and sulphonates, sulphated monoglycerides, sulphated ethers, sulphosuccinates, alkane sulphonates, phosphate esters, alkyl isethionates, and sucrose esters. Preferred anionic detergents include sodium dodecyl sulfate (SDS) and lithium dodecyl sulfate. Cationic detergents may alternatively be employed in the protease-based lysis buffer of the invention. Examples of cationic detergents include the quaternary ammonium salts (e.g., cetyl trimethylammonium chloride).

The preferred detergents for use in the protease-based lysis buffer of the invention are ionic detergents. Such detergents are able to disrupt intermolecular interactions (e.g., the binding of proteins to nucleic acid molecules) and to inactivate proteins (e.g., by precipitation/aggregation or through denaturation). The inclusion of one or more of these detergents in the protease-based lysis buffer of the invention, then, contributes to the inactivation of cellular nucleases (thus protecting cellular nucleic acid molecules from degradation), as well as decreasing the ability of cellular proteins to associate with the cellular nucleic acid molecules (thus increasing the accessibility of the cellular nucleic acid molecules to the oligonucleotide primer and probe molecules of the invention). Also, the particularly preferred ionic detergent of the invention, SDS, enhances the activity of the preferred protease of the invention, proteinase K, thereby increasing the degradation of cellular proteins which may interfere with the stability or accessibility of cellular nucleic acid molecules in the sample.

2. Protease

To degrade proteins associated with cellular nucleic acid molecules which may interfere with nucleic acid unpackaging or unfolding, nucleic acid strand separation, or hybridization of oligonucleotide primers to one or more target sequences of the nucleic acid molecules, or to degrade proteins which themselves degrade nucleic acid molecules, a protease is an essential component of the protease-based lysis buffer. This protease is preferably nonspecific, such that the preponderance of the proteins in the cellular lysate may be degraded by its enzymatic action. Further, preferred proteases are enzymatically active at an environmental condition at which the cellular nucleic acid molecules are not damaged. For example, acidic conditions are known to partially depurinate the DNA, leading to strand cleavage. However, nucleic acid molecules are known to be generally tolerant to changes in temperature; at temperatures above 95 degrees, double-stranded DNA will separate into single strands, due to disruption of the interstrand hydrogen bonds, but once the temperature is lowered, the strands re-anneal. Thus, the temperature at which the protease functions should have little effect on the stability of cellular nucleic acid molecules in the sample. One benefit of using a protease with an optimal temperature range higher or lower than about 37° C. is that there is a good possibility that cellular nucleases may be decreased in activity at such temperatures. Lastly, the preferred protease of the invention is preferably easily inactivated, such that inhibitory agents or harsh environmental conditions which might damage cellular nucleic acid molecules are avoided.

The preferred protease of the invention is proteinase K. The preferred temperature at which proteolysis is carried out is between about 37° C. and about 65° C., more preferably between about 45° C. and 55° C., and most preferably about 50° C. The most preferred temperature at which proteolysis is carried out is that at which the cellular nucleic acids are substantially protein-free at the end of the protease treatment.

3. Buffering Agent

A buffering agent is an essential part of the protease-based lysis buffer of the invention, not only to maintain the pH of the lysate such that the cellular nucleic acids are not damaged (for example, to avoid the depurination of nucleic acid molecules which occurs at acidic pH), but also to maintain a pH at which the protease of the lysis buffer is most active. It will be recognized by one skilled in the art that buffering agents are typically temperature-sensitive, and thus the temperature at which the proteolysis step will be conducted must be considered in the selection of an appropriate buffering agent and pH thereof. Many such buffering agents and their buffering capacities at different temperatures are known to those skilled in the art. A preferred buffering agent for use in the invention (for use in a lysis buffer containing Proteinase K) is Tris base. A preferred pH range for the lysis buffer at 50° C. is about pH 7.0 to about pH 8.0; a more preferred pH range for the lysis buffer at 50° C. is about pH 7.3 to about pH 7.7; a particularly preferred pH for the lysis buffer at 50° C. is about pH 7.5.

4. Other Components

The protease-based lysis buffer of the invention is preferably free of compounds which are inhibitory to a subsequent amplification reaction. Such compounds include, but are not limited to, chaotropic salts (e.g., LiCl), metal ions (e.g., Mg²⁺), and phenol or chloroform. The absence of these compounds permits protein digestion and subsequent amplification of one or more target sequences from the nucleic acid molecules of the cell without further purification steps, thereby substantially reducing sample loss and contamination.

It will be appreciated by one skilled in the art that it is possible to assess whether or not a given compound is inhibitory or stimulatory to proteolysis by the selected protease or to subsequent amplification of one or more selected cellular nucleic acid molecules prepared through the use of the protease-based lysis buffer of the invention. Such an analysis is performed by including the compound in question in the lysis buffer and comparing proteolysis and/or amplification of the test sample to control reactions lacking the compound.

Alkaline Lysis Buffer Without DTT

The methods of the invention also provide an alkaline lysis buffer that uses an alkaline lysis protocol known in the art (e.g., the procedure of Cui, as modified in Gitlin, S. A. et al. (1996) J. Assist. Reprod. Genet. 13:107-111), except that the reagent dithiothreitol (DTT) is omitted. While standard alkaline lysis protocols that include DTT result in amplification, the efficiency of amplification is lower than that observed with the protease-based lysis buffer of the invention. Omission of DTT from the alkaline lysis buffer results in successful amplification, comparable to that seen with the protease-based lysis buffer.

Methods for Cellular Lysis and Nucleic Acid Sample Preparation from a Cell

The invention provides a method for the preparation of substantially accessible nucleic acid molecules from a cell. This method consists of treating a cell with the protease-based lysis buffer of the invention to form a mixture, and incubating this mixture under conditions in which cellular proteins are substantially degraded by the protease of the lysis buffer, such that substantially accessible nucleic acid molecules are obtained. Optionally, a protease may be selected which is operative at a temperature at which cellular nucleases are largely inactive, to limit degradation of the nucleic acid molecules.

The invention also provides a method for the preparation of substantially accessible nucleic acid molecules from a single cell or a part thereof (e.g., an organelle). This method consists of treating a single cell or part thereof with the protease-based lysis buffer of the invention to form a mixture, and incubating this mixture under conditions in which cellular proteins are substantially degraded by the protease of the lysis buffer while cellular nuclease activity is substantially inhibited, such that substantially accessible nucleic acid molecules are obtained.

The invention also provides a method for preparing nucleic acid molecules from a cell for an amplification reaction. This method consists of treating a cell with the protease-based lysis buffer of the invention (which is lacking in components which are inhibitory to an amplification reaction such as the polymerase chain reaction, as discussed herein) to form a mixture, and incubating this mixture under conditions in which cellular proteins are substantially degraded by the protease of the lysis buffer, such that substantially accessible nucleic acid molecules are obtained which may be directly utilized in an amplification reaction.

The invention also provides a method for preparing nucleic acid molecules from a single cell or part thereof (e.g., an organelle) for an amplification reaction. This method consists of treating a single cell or part thereof with the protease-based lysis buffer of the invention (which is lacking in components which are inhibitory to an amplification reaction such as the polymerase chain reaction, as discussed herein) to form a mixture, and incubating this mixture under conditions in which cellular proteins are substantially degraded by the protease of the lysis buffer while cellular nuclease activity is substantially inhibited, such that substantially accessible nucleic acid molecules are obtained which may be directly utilized in an amplification reaction.

The treatment step of the method may be conveniently performed in any standard reaction vessel, preferably one having a capacity commensurate with the volume of the sample (e.g., a sealable microcentrifuge tube or a microtiter plate), and one which permits the direct detection of the label associated with the primer or probe (e.g., an optically clear tube for the detection of a fluorescent signal). It is preferred that the reaction vessel is treated such that the surface contacting the sample is decreased in affinity for the nucleic acid molecules of the invention. It is also preferred that the reaction vessel be of a configuration to permit ease of inclusion in a thermal cycler apparatus such that an amplification reaction may subsequently be performed. It is particularly preferred that the reaction vessel be tightly sealable or otherwise contained such that contamination of the sample or the surroundings is minimized.

The incubation step of the method is at an environmental condition and for a period of time commensurate with the activity of the selected protease. In the preferred embodiment in which the lysis buffer contains proteinase K, the incubation step is conducted at greater than about 37° C., preferably at between about 37° C. and about 65° C., more preferably at between about 42° C. and about 55° C., and most preferably at about 50° C. The time of incubation is also dependent on the particular protease selected. In the preferred embodiment in which the lysis buffer contains proteinase K, the incubation step is conducted for a period of time between about 10 minutes and about 90 min, more preferably between about 30 min and about 75 min, and even more preferably for about 60 min. Appropriate methodologies by which the optimal temperature (or other environmental condition) and time of incubation can be experimentally determined for any given protease (where this information is not already readily available) are well known to those skilled in the art. The most preferred incubation conditions are those which result in the preparation of substantially protein-free (and therefore accessible to oligonucleotide primer and probe molecules) cellular nucleic acid molecules.

It will be appreciated by one skilled in the art that a further step in which the protease of the lysis buffer is inactivated may be desired in the method. Such a step may be required in circumstances where the protease does not self-inactivate through self-proteolysis and in which further manipulation of the sample through protein action (e.g., an amplification reaction involving a polymerase molecule) is required. Such inactivation steps frequently are readily achieved by a brief high-temperature incubation, resulting in denaturation of all proteins in the sample. In the preferred embodiment in which the lysis buffer contains proteinase K, the inactivation step is conducted at about 95° C. for a period of about 10 minutes. Inactivation means (e.g., temperature inactivation or the introduction of protease inhibitors) and/or the methods for determining such means for different proteases utilized in the invention are well known to those skilled in the art.

II. Moderately-Repeated Highly-Conserved Nucleic Acid Sequences

In order to identify the presence of a selected nucleic acid molecule (e.g., a chromosome) in a sample containing nucleic acid molecules, it is possible to amplify a specific single-copy gene known to be located in the selected nucleic acid molecule. The presence of an amplicon corresponding to the target gene at the end of an amplification reaction indicates the presence of the selected nucleic acid molecule. However, this method of detection suffers from the fact that an amplification reaction (e.g., the polymerase chain reaction) is multiplicative in nature (in that each amplicon itself may serve as a template for a subsequent replication step), and one or more rounds of amplification in which primer annealing does not occur may result in substantial decreases in the amplified product resulting from the overall amplification process. The utilization of a single copy gene as a target sequence renders the likelihood of a skipped amplification initiation event high, since there would be very few primer annealing sites present in the sample.

Even given these limitations, however, the methods and compositions of the invention permit the detection and/or quantification of a selected nucleic acid molecule (e.g., a chromosome) through the amplification of a single copy gene of that selected nucleic acid molecule. For example, the protease-based lysis buffer of the invention permits the preparation of highly accessible (e.g., protein-free) nucleic acid molecules from the cell, such that a skipped amplification initiation event due to the presence of a protein bound to the nucleic acid is less likely. Further, the methods and compositions of the invention also increase the sensitivity of detection of the desired nucleic acid molecules through decreased sample contamination and loss, such as by utilizing a lysis buffer which does not interfere with a subsequent amplification reaction, such that purification of the cellular nucleic acids is not required. Thus, the methods of the invention are also commensurate with the amplification of a single-copy gene for the detection and/or quantification of a selected nucleic acid sequence.

Alternatively, it is possible to target highly repeated nucleic acid sequences characteristic of the selected nucleic acid molecule for amplification in order to detect the presence of this selected nucleic acid molecule. Highly repeated nucleic acid sequences offer an increased number of target sequences, such that a skipped amplification initiation event for any one target sequence is of less consequence to the overall detectability of the amplicon at the end of the overall amplification reaction. However, these highly repeated sequences frequently are not well-conserved, such that oligonucleotide primers specific for one of the target highly repeated sequences may not hybridize well to another copy of the same highly repeated sequence. Thus, although there exist large numbers of repeats of the sequence within the selected nucleic acid molecule, only a fraction of these repeats may be detected with any two primers. Further, these highly-repeated sequences are frequently present on more than one chromosome, rendering it difficult to specifically detect only a single chromosome in a sample.

The compositions of the invention provide an improved alternative to either the single-copy or highly repeated sequences in the form of moderately-repeated highly-conserved sequences. These sequences are found in greater than three copies within a given nucleic acid molecule (or throughout the genetic complement of a cell), and are selected on the basis of their specificity for one or more target nucleic acid molecules. Further, unlike highly repeated sequences, the moderately-repeated highly-conserved sequences of the invention are sufficiently conserved such that the oligonucleotide primer and probe molecules which hybridize to one instance of the moderately-repeated highly-conserved sequence will also hybridize to a plurality of the other copies of the sequence present in the target nucleic acid molecule under stringent hybridization conditions. The moderately-repeated highly-conserved sequences of the invention may be conveniently identified through a survey of genetic databases for a selected organism.

Nucleotide Molecules of the Invention

In one aspect, the invention provides isolated nucleic acid molecules comprising a moderately-repeated highly-conserved sequence, and oligonucleotide fragments which may be utilized as amplification primers or as hybridization probes for these moderately-repeated highly-conserved sequences.

Two such isolated moderately-repeated highly-conserved nucleic acid molecules, in particular, are provided: the U2 and TSPY genes. The TSPY gene is repeated 27-40 times within clusters on the human Y chromosome (Zhang, J. S. et al. (1992) Hum. Mol. Genet. 1:717-726; Manz, E. et al. (1993) Genomics 17:726-731), and the U2 sequence is repeated 10-20 times on human chromosome 17 (Van Arsdell, S. W. and Winer, A. M. (1984) Mol. Cell Biol. 4:492-499; Westin, G. et al. (1984) Proc. Natl. Acad. Sci. U.S.A. 81:3811-3815; Pavelitz, T. et al. (1995) EMBO J. 14:169-177). The TSPY nucleotide sequence may be found at least for example in GenBank Accession No. M98524, and the U2 nucleotide sequence may be found at least for example in GenBank Accession No. L37793.

A nucleic acid molecule of the present invention, e.g., a nucleic acid molecule having a sequence corresponding to that of a moderately-repeated highly-conserved sequence, or a portion thereof, can be isolated using standard molecular biology techniques (see, for example, Ausubel, F. et al. Current Protocols in Molecular Biology (1999) J. Wiley: New York; and Sambrook et al. Molecular Cloning: A Laboratory Manual, 2^{nd} ed., (1989) Cold Spring Harbor Laboratory Press: Cold Spring Harbor, N.Y.). Using all or a portion of a moderately-repeated highly-conserved sequence as a hybridization probe, nucleic acid molecules containing these repeat sequences may be isolated using standard hybridization and cloning techniques.

Moreover, a nucleic acid molecule encompassing all or a portion of a moderately-repeated highly-conserved sequence can be isolated by an amplification reaction (e.g., the polymerase chain reaction) using synthetic oligonucleotide primers designed based upon the sequence of a desired moderately-repeated highly-conserved sequence. A nucleic acid of the invention can be amplified using cDNA, mRNA or alternatively, genomic DNA, as a template and appropriate oligonucleotide primers according to standard amplification techniques.

Oligonucleotides complementary to a moderately-repeated highly-conserved nucleotide sequence can be prepared by standard synthetic techniques, for example, by using an automated DNA synthesizer.

The oligonucleotide probe and primer molecules complementary to the moderately-repeated highly-conserved sequences of the invention typically comprise only a portion of the nucleic acid sequence of these repeated regions. These oligonucleotide molecules generally are substantially purified and free of contaminating material. The oligonucleotide molecules typically comprise a region of nucleotide sequence that hybridizes under stringent conditions to at least about 12 or 15, and more preferably to at least about 20, 25, 30, 35, 40, 45, 50, 55, 60, 65, or 75 consecutive nucleotides of a sense or antisense sequence of a moderately-repeated highly-conserved sequence. The preferred oligonucleotide primers of the invention hybridize stringently to about 18-20 consecutive nucleotides of a sense or antisense sequence of a moderately-repeated highly-conserved sequence. The preferred oligonucleotide probe molecules of the-invention hybridize stringently to about 15 to 30 consecutive nucleotides of a sense or antisense sequence of a moderately-repeated highly-conserved sequence.

In another embodiment, the moderately-repeated highly-conserved nucleic acid molecules and oligonucleotides of the present invention can be modified at the base moiety, sugar moiety or phosphate backbone to improve, for example, the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, B. et al. (1996) supra and Perry-O'Keefe et al. (1996) Proc. Natl. Acad. Sci. USA 93:14670-675.

Detectable Labels

The invention also provides detectably labeled oligonucleotide primer and probe molecules. Typically, such labels are chemiluminescent, fluorescent, radioactive, or colored, or consist of an enzymatic moiety that can produce a chemiluminescent, fluorescent, radioactive, or colored signal upon incubation with an appropriate substrate to permit ease of detection. Such labels, appropriate detection methods, and the criteria by which one label would be selected over another are well known to those skilled in the art. For use in the detection of an amplified target sequence during an amplification reaction, preferred labels are those which may be detected without the addition of substrates (which may interfere with the progression of the amplification reaction), those which may be detected rapidly (such that the quantity or presence of amplicon may be measured at each selected time (e.g., cycle) of an amplification reaction without stopping the reaction), and those which may be detected without additions to or modifications of the sample (e.g., the reaction vessel need not be opened), such that no contamination of the sample or the surroundings takes place.

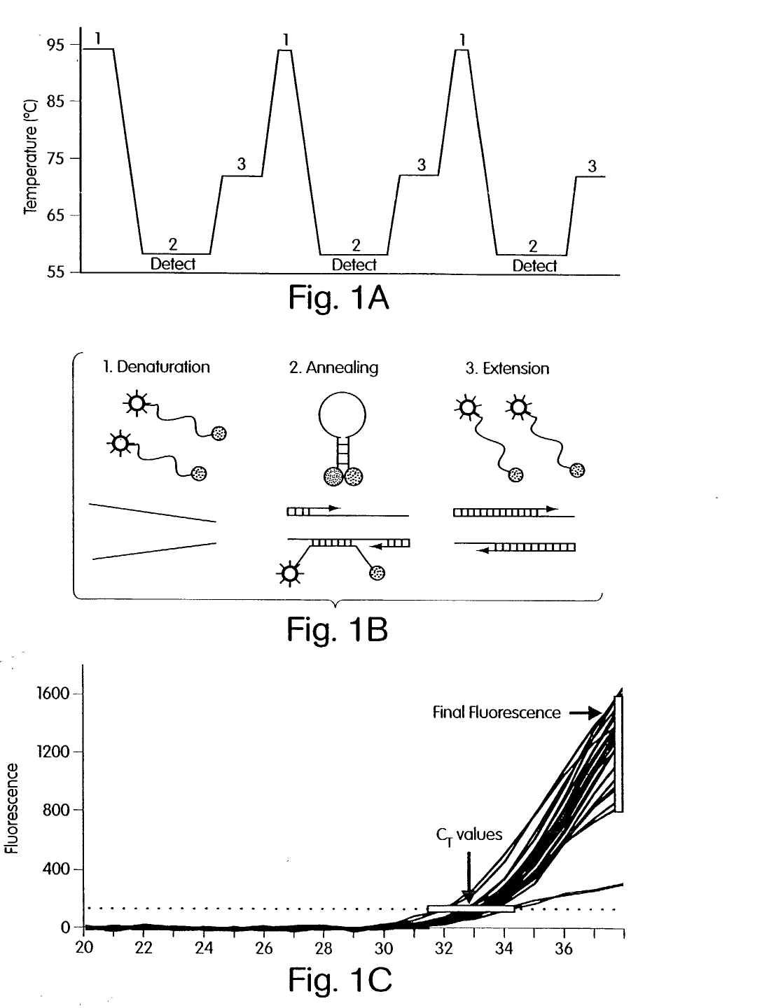

One variety of detectable label which is particularly well-suited to the methods of the invention is a molecular beacon. The invention therefore includes molecular beacon oligonucleotide primer and probe molecules having at least one region which is complementary to a moderately-repeated highly-conserved nucleic acid of the invention, such that the molecular beacon is useful for quantitating the presence of moderately-repeated highly-conserved nucleic acid of the invention in a sample. A “molecular beacon” (see, e.g., Tyagi and Kramer (1996) Nat. Biotechnol. 1:151-6; Lizardi et al., U.S. Pat. No. 5,854,033; Nazarenko et al., U.S. Pat. No. 5,866,336; and Livak et al., U.S. Pat. No. 5,876,930) is a single-stranded oligonucleotide 25-35 bases long in which the last 5-8 bases on the 3′ and 5′ ends are complementary (see FIG. 1B). Thus, a beacon forms a hairpin structure at ambient temperatures. The double-helical stem of the hairpin brings a fluorophore attached to the 5′ end of the beacon very close to a quencher attached to the 3′ end of the beacon (see FIG. 1B). The beacon does not fluoresce in this conformation. However, if the beacon is heated or allowed to hybridize to a target oligonucleotide which is complementary to the sequence within the single-strand loop of the beacon, the fluorophore and the quencher are separated and the resulting conformations fluoresce. Thus, when fluorescent readings are acquired at the annealing temperature at a series of selected times during an amplification reaction, the signal strength increases in proportion to amplicon accumulation. Molecular beacons with different loop sequences can be constructed with different fluorophores in order to monitor increases in different amplicons in multiplex reactions (Tyagi et al. (1998) Nat. Biotechnol. 16:49-53; Kostrikis et al. (1998) Science 279:1228-1229). As discussed herein, the preferred molecular beacon oligonucleotide of the invention is one sufficiently complementary such that a single base pair mismatch prevents hybridization under the stringent hybridization conditions of the invention.

Molecular beacon-tagged oligonucleotide molecules which hybridize specifically to the moderately-repeated highly-conserved sequences of the invention therefore permit not only detection of the label only in the instance where the oligonucleotide molecule bearing the molecular beacon either is or is not hybridized to a target sequence, but also detection of hybridization without additions to or modifications of the sample (e.g., the reaction vessel can remain sealed). This latter point is particularly important: not only is possible contamination of the sample minimized or eliminated through use of this type of detectable label, but real-time monitoring of amplicon accumulation is possible due to the ease of detection of the amplicon.

Furthermore, it is possible to utilize more than one detectably labeled primer or probe simultaneously in the methods of the invention. In such a situation, two or more different detectable labels may be used, wherein the different detectable labels are separately detectable (e.g., fluorescent labels which emit light at different wavelengths). Appropriate labels and combinations thereof in which the individual labels may be separately detected are known to those skilled in the art.

It should be understood that the detection of the label is direct evidence of only the labeled oligonucleotide primer or probe (and in preferred situations, direct evidence of these oligonucleotide molecules in either the hybridized or unhybridized state). The presence of the selected nucleic acid molecule in the reaction is inferred from such binding, since the nucleic acid sequence for which the labeled primer or probe is specific has been selected due to the fact that it is localized to the selected nucleic acid molecule. In order to ensure the validity of this inference, appropriate control reactions must be conducted (e.g., in which the ability of the oligonucleotide primer/probe molecules to hybridize to the target sequence on the selected nucleic acid molecule under similar reaction conditions is assessed, and in which the specificity of the oligonucleotide primer/probe molecules for the target sequence is assessed).

Enhancers of Molecular Beacon Probes

It is known that only a minority of the molecular beacon probe molecules added to an amplification reaction for amplicon detection actually bind to their targets. In the beginning of a reaction, this happens because the total number of probes is in vast excess of the number of amplicons. Toward the end of the reaction, it happens because the separate strands of the amplicon hybridize to each other, preventing or displacing most of the probe molecules bound to the target strands. It therefore is possible to enhance the molecular beacon signal by exposing or keeping the strand of an amplicon open (e.g., in a single-stranded or unhybridized state) in the region where the molecular beacon probe hybridizes to its target sequence. The resulting increase in local single-strandedness at a temperature low enough to permit molecular beacon/target interactions can increase the percentage of bound molecular beacon molecules and would therefore enhance the intensity of the signal. Increased signal intensity is preferable for detection of signals that require so many cycles of amplification that they risk exhausting the amplification capacity of the reaction.

In one embodiment, an enhancer of a molecular beacon probe is an oligonucleotide or modified oligonucleotide (e.g., an oligonucleotide that contains 2′O-methyl bases or peptide nucleic acids) that hybridizes to the sequences flanking-the target site of the molecular beacon probe and thereby causes formation of a D-loop that contains the molecular beacon target sequence. Oligonucleotide enhancers are not degraded or incorporated in the amplification process and do not act as primers. Oligonucleotide enhancers may temporarily inhibit synthesis of a new template strand, but the enhancers are designed to fall off the template strand when the elongation step of the reaction is carried out at an elevated temperature. Oligonucleotide enhancers can be used singly or in pairs, and their length and hybridization specificity can be adjusted to provide the desired characteristic of temporary D-loop formation.

In other embodiments, an enhancer of a molecular beacon probe is a protein (e.g., a non-enzymatic protein or an enzyme) which either binds to or binds to and degrades sequences near or complementary to the target sequence, without binding to or degrading the target sequence itself, thus rendering the target sequence single-stranded and available for hybridization with the molecular beacon probe. Protein enhancers may temporarily inhibit synthesis of a new template strand, but the enhancers are designed to fall off the template strand when the elongation step of the reaction is carried out at an elevated temperature.

In a preferred embodiment, enhancers of molecular beacon probes, including oligonucleotide and protein enhancers, are added an amplification reaction before the reaction is commenced. However, enhancers may also be added to a completed amplification reaction in order to increase the intensity of the final signal.

Best-Possible Primers

While most applications of PCR technology focus on amplification of a specific amplicon, methods for selecting and optimizing primers have not been systematically described. Sets of primers are typically designed by use of appropriate software that helps define short oligomeric sequences that are least likely to cross-hybridize with each other or with inappropriate sites in a genome, to the extent that such sites are known from sequence data. The selected pairs of primers are then typically tested in a PCR reaction containing 10 or more copies of the target sequence in one or more genomes, and the products are analyzed for the presence of specific amplicon and nonspecific amplicon products. If the specific amplicon is the dominant product generated in the PCR reaction, the pair of primers is typically declared sufficiently reliable for clinical and research use.

However, analysis of PCR initiated with fewer than 10 copies of the target sequence demonstrates that oligonucleotide primer sets that appear to reliably generate specific amplicon products in the presence of 10 or more copies of the target sequence may generate amplification of nonspecific sequences in reactions initiated with fewer than 10 copies of the target sequence. The reason for this phenomenon may be the statistical probability of amplifying nonspecific amplicons. The present invention provides a method for selecting primers (best-possible primers, or BP primers) that maximize specific amplicon amplification by examining the occurrence of nonspecific amplicon amplification. BP primers are those that generate the fewest nonspecific amplicons while not negatively impacting amplification of specific amplicons.

Selection of BP Primers via Amplification Using Fewer Than 10 Starting Target Sequences

Oligonucleotide primers to be tested can be designed as described above, and may be tested first using GD-DNA (see below). For each primer pair, replicate assays (2 or more, and preferably 5 or more) are carried out using the amplification methods of the invention (e.g., PCR), and the amplicon products are detected (e.g., using SYBR® Green, or an amplicon specific fluorescent probe (e.g., a molecular beacon)). The target sequence is present in fewer than 10 copies, 5 or fewer copies, 2 or fewer copies, or only one copy. The results may be analyzed with respect to the magnitude of the resulting C_{T} values; the variance of the C_{T} values; the magnitude of the fluorescence reached 4-6 cycles beyond the C_{T} value; the variance of the fluorescence 4-6 cycles beyond the C_{T} value. The results can also be judged in terms of the rate of signal increase. In a preferred embodiment, BP primers have at least one of the following properties: lower C_{T} values, smaller C_{T} value variance, higher fluorescence 4-6 cycles beyond the C_{T} value, smaller variance of the fluorescence 4-6 cycles beyond the C_{T} value, a greater rate of signal increase, and fewer non-specific amplicons than other primer pairs specific for the same target sequence.

BP primers and optimal reaction conditions can also be established by examination of the hybridization melting curves of the resulting amplicons. It is preferable to achieve sharp melts that display a single peak of the predicted melting values.

Variables that may be tested in the amplification methods used to test the primers sets include: Mg²⁺ concentration, K⁺ concentration, temperature, pH, buffer concentration, primer concentration, and deoxynucleotide concentration.

Gene-Deleted DNA

BP primers may also be selected using Gene-Deleted DNA (GD-DNA). GD-DNA contains all, or nearly all, of the DNA sequences present in Complete Genome DNA (CG-DNA), but is unable to replicate or amplify selective sequences in the CG-DNA. In most cases, GD-DNA is prepared from CG-DNA by chemical or biochemical treatment. DNA from a homozygous knockout source (e.g., a knockout animal such as a mouse) or from a source that lacks a specific chromosome can be regarded as a special type of GD-DNA that is either naturally occurring or man-made.

GD-DNA can be prepared from CG-DNA by treating the CG-DNA with a sequence-specific replication inhibitor (SSRI) that prevents primary replication of a particular sequence, or family of closely-related sequences, and may also prevent further amplification of said sequence(s). There are many types of SSRIs, including, but not limited to, restriction enzymes, oligonucleotides, and protein nucleic acids (PNAs) with or without use of a single strand nuclease. It should be understood that one skilled in the art will be capable of using or combining these or other SSRIs to generate GD-DNA for specific sequences.

GD-DNA can be used to select BP primers for a target sequence by performing an amplification reaction using the methods described herein with GD-DNA as a template, said GD-DNA being deleted for the target sequence. BP primers are those that generate the fewest nonspecific amplicons while not negatively impacting the generation of specific amplicons when the same primers are used in an amplification reaction using CG-DNA as a template.

The amplification reaction using GD-DNA as a template preferably will contain multiple genomes (e.g., at least 10-10,000 genomes) of GD-DNA. Each set of primers is tested in at least 2, and preferably at least 5 or more replicate assays for about 45 cycles. Products can be analyzed, for example, by gel electrophoresis and stained, for example, with SYBR® Green. All products generated using GD-DNA as a template will be nonspecific amplicons, likely due to primer-dimer formation and amplification or hybridization of primers to non-specific sites in the genome.

When a primer pair is identified as potential BP primers, it may by tested in further amplification reactions with fewer than 10 target sequences, 5 or fewer target sequences, 2 or fewer target sequences, or only one target sequence of CG-DNA (see above) to determine optimal reaction conditions.

III. Methods for Detecting the Presence or Quantity of a Selected Nucleic Acid Molecule in a Sample Containing Nucleic Acids

In one embodiment, the invention provides a method for detecting the presence or quantity of a target nucleic acid molecule (e.g., a chromosome) or portion thereof in a sample containing nucleic acid molecules. In this method, a moderately-repeated highly-conserved sequence found within the target nucleic acid molecule is amplified (e.g., by a polymerase chain reaction) using at least two oligonucleotide primer molecules sufficiently complementary to opposite strands of the moderately-repeated highly-conserved molecule such that these primer molecules are able to hybridize with a plurality of the copies of the moderately-repeated highly-conserved sequence present in the sample. The amplified moderately-repeated highly-conserved nucleic acid sequence is detected as indicative of the presence or quantity of the target nucleic acid molecule or portion thereof in the sample. In a preferred embodiment, this detection step measures the detectable label associated with at least one of the oligonucleotide primers. In another preferred embodiment, this detection step takes place at selected times (e.g., cycles) of amplification by measuring the amount of the labeled primer hybridized to the moderately-repeated sequence.

In another embodiment, the invention provides another method of detecting the presence or quantity of a nucleic acid molecule (e.g., a chromosome) or portion thereof in a nucleic acid sample. In this method, the sample is contacted with at least two nucleic acid primers, each primer being sufficiently complementary to an opposite strand of a moderately-repeated highly-conserved nucleic acid sequence found within the nucleic acid molecule (e.g., the chromosome) or portion thereof such that they are able to hybridize with a plurality of these sequences and prime the amplification of this target sequence. The sample is also contacted with at least one detectably labeled probe which is sufficiently complementary to the above-mentioned moderately-repeated highly-conserved nucleic acid sequence such that it hybridizes to a plurality of the copies of the sequence present in the sample. The moderately-repeated highly-conserved nucleic acid sequence is amplified by an amplification reaction, and the amplified moderately-repeated highly-conserved nucleic acid sequence is detected at selected times (e.g., cycles) of amplification by measuring the label associated with the probe hybridized to a plurality of the copies of the nucleic acid sequence present in the reaction.

In another embodiment, the invention provides a process for detecting and/or quantifying a nucleic acid of interest from a group of fewer than 10 cells, 5 or fewer cells, 2 or fewer cells, or a single cell or a part thereof (e.g., an organelle), in which a lysis buffer provided by the invention is used to lyse the group of cells, the single cell or the part thereof according to the methods described herein, an amplification reagent which specifically amplifies a moderately-repeated highly-conserved sequence of the target nucleic acid molecule (e.g., the chromosome) is added to the nucleic acid sample, and the moderately-repeated highly-conserved sequence is amplified. The amplified sequence is detected through either the use of a detectably labeled primer or a detectably labeled probe also included in the amplification reagent which specifically hybridize to a plurality of the copies of the amplified moderately-repeated highly-conserved sequence in the sample, in which the detectable label is detectable without additions to or modifications of the sample (e.g., without opening the reaction vessel).

In another embodiment, the invention further provides a process for detecting and/or quantifying a nucleic acid of interest from a group of fewer than 10 cells, 5 or fewer cells, 2 or fewer cells, or a single cell or a part thereof (e.g., an organelle) in one reaction vessel, in which a lysis buffer provided by the invention is used to lyse the group of cells, the single cell, or the part thereof in a reaction vessel according to the methods described herein, an amplification reagent which specifically amplifies a moderately-repeated highly-conserved sequence of the target nucleic acid molecule (e.g., the chromosome) is added to the nucleic acid sample, and the moderately-repeated highly-conserved sequence is amplified. The amplified sequence is detected through either the use of a detectably labeled primer or a detectably labeled probe also included in the amplification reagent which specifically hybridizes to a plurality of the copies of the amplified moderately-repeated highly-conserved sequence in the amplification reaction.

In a further embodiment, the invention provides a method for determining the presence or quantity of human chromosome 17 in a group fewer than 10 human cells, 5 or fewer human cells, 2 or fewer human cells, a single human cell, or a part of a human cell (e.g., a nucleus) in which a lysis buffer provided by the invention is used to lyse the group of human cells, the single human cell, or the part thereof according to the methods described herein, an amplification reagent which specifically amplifies a moderately-repeated highly-conserved sequence of human chromosome 17 (e.g., the U2 sequence) is added to the nucleic acid sample, and the moderately-repeated highly-conserved sequence is amplified. The amplified sequence is detected through either the use of a detectably labeled primer or a detectably labeled probe also included in the amplification reagent which specifically hybridizes to a plurality of the copies of the moderately-repeated highly-conserved sequence from human chromosome 17, in which the detectable label is detectable without additions to or modifications of the sample (e.g., without opening the reaction vessel).

In a further embodiment, the invention provides a method for determining the sex of a group of fewer than 10 human cells, 5 or fewer human cells, 2 or fewer human cells, a single human cell, or a part thereof (e.g., a nucleus) in which a lysis buffer provided by the invention is used to lyse the group of human cells, the single human cell, or the part thereof according to the methods described herein, an amplification reagent which specifically amplifies a moderately-repeated highly-conserved sequence of the human Y chromosome (e.g., the TSPY sequence) is added to the nucleic acid sample, and the moderately-repeated highly-conserved sequence of the human Y chromosome is amplified. The amplified sequence is detected through either the use of a detectably labeled primer or a detectably labeled probe also included in the amplification reagent which specifically hybridizes to a plurality of the copies of the moderately-repeated highly-conserved sequence from the human Y chromosome, in which the detectable label is detectable without additions to or modifications of the sample (e.g., without opening the reaction vessel).

It should be noted that the methods and compositions of the invention permit the discrimination between as few as one and two copies of a selected nucleic acid molecule in a sample, as is demonstrated in the example section.

The amplification and detection steps involved in these methodologies are discussed further below.

Amplification

An amplification reaction is composed of a series of steps, resulting in the synthesis of a target sequence in a geometric or exponential fashion. The steps involved in a typical cyclical amplification reaction (e.g., the polymerase chain reaction) include: denaturation of the template nucleic acid molecule to result in single-stranded nucleic acid molecules; annealing of the primers specific for the target nucleic acid sequence to the target nucleic acid sequence, such that (ideally) each copy of the nucleic acid sequence is hybridized to a primer, and synthesis of a strand complementary to the target sequence, using the primer to prime synthesis, a polymerase, a buffering agent, and the deoxynucleotide triphosphate molecules present in the amplification reagent. A subsequent denaturation step separates all double-stranded nucleic acid molecules, and the newly-synthesized template strands may also serve as template sequences for a subsequent round of nucleic acid synthesis, resulting in an exponential increase in the amount of amplicon in the reaction. Conditions, reagent concentrations, primer design, and appropriate apparatuses for typical cyclic amplification reactions are well known in the art (see, for example, Ausubel, F. Current Protocols in Molecular Biology (1988) Chapter 15: “The Polymerase Chain Reaction”, J. Wiley: New York).

There exist numerous variations on the typical amplification reaction which may be adapted to the methods of the invention. The polymerase chain reaction (PCR) is described in U.S. Pat. Nos. 4,683,195 and 4,683,202. Alternatively, a ligation chain reaction (LCR) may be used (see, e.g., Friedhoff, P. et al. (1988) Science 241:10771080; and Nakazawa et al. (1994) Proc. Natl. Acad. Sci. USA 91:360-364). rtPCR (Mullis (1987) U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), and Q-Beta amplification (Lizardi et al. (1988) Bio/Technology 6:1197).

In one embodiment, the invention provides a method for cell lysis and nucleic acid amplification (e.g., by PCR) in a single tube. Analysis of cellular nucleic acids using PCR requires the separation of proteins and other cellular components from the DNA. However, all published methods of cellular lysis and PCR require that the sample tube be opened and the reagents for PCR added only after the lysis step is complete, even when both lysis and PCR are done in the same tube. Eliminating the extra step of PCR reagent addition would reduce the time needed for the assay to be completed and would reduce the possibility of contamination. When working with small numbers of cells (e.g., single cells), even extremely small amounts of contamination can interfere with interpretation of results. Therefore, it is highly preferable to minimize contamination wherever possible.

Most of the buffering conditions used for the protease incubation of the invention are compatible with PCR amplification. Furthermore, reagents such as deoxynucleotides, oligonucleotide primers, and oligonucleotide probes (e.g., molecular beacon probes) are not measurably affected by incubation with a protease. However, the polymerase cannot be present in the lysis solution since it would be degraded by the protease. Also, it is preferable to avoid adding magnesium to the lysis solution, since it results in suboptimal lysis, possibly by inhibition of the protease.

The method provided by the present invention includes adding all the reagents required for PCR to the lysis solution prior to the lysis incubation step. The polymerase and the magnesium are added in a form in which they cannot contact the lysis solution. In a preferred embodiment, the polymerase and magnesium are encased in wax beads, preferably separate from each other. Wax beads containing Taq polymerase are commercially available from, for example, Promega. Wax beads containing magnesium are commercially available from, for example, Stratagene. The wax prevents contact between the polymerase and magnesium and the other components of the reaction. The polymerase and magnesium are then released into solution following the lysis incubation and prior to the amplification step by incubating the reaction at a temperature sufficient to both melt the wax and inactivate any protease in the lysis buffer. Such a sufficient temperature is at least about 90° C. and preferably is at least about 95° C. Amplification of the target sequences is then possible.

Aside from cyclic amplification reactions, there also exist non-cyclic amplification reactions, such as rolling circle amplification (Lizardi et al. U.S. Pat. No. 5,854,033 and Lizardi et al. (1998) Nat. Genet. 19:225-232), which may be readily used in the methods of the invention. The steps involved in rolling circle replication include: denaturation of the template nucleic acid molecule to result in single-stranded nucleic acid molecules; annealing of a first oligonucleotide primer specific for a target nucleic acid sequence to that target nucleic acid sequence such that both the 5′ and the 3′ ends of the primer anneal to the target nucleic acid sequence simultaneously; filling in of the gap between the 5′ and 3′ ends of the primer by ligation, by binding and ligation of a small phosphorylated nucleotide, or by synthesis of the intervening sequence using the target nucleic acid sequence to which the primer is annealed as a template and an amplification reagent including a polymerase, dNTPs, a salt, and a buffering agent such that the annealed primer now forms a closed circle tethered to the target nucleic acid sequence; annealing of a second primer to a sequence of the first primer other than that already hybridized to the target sequence; and synthesis of a nucleic acid strand complementary to that of the circularized first primer, using the second primer to prime synthesis, a strand-displacing polymerase, a buffering agent, and the deoxynucleotide triphosphate molecules present in the amplification reagent. The result of this amplification is a long, single-stranded nucleic acid molecule consisting of tandem repeats of the circularized first primer. Since each repeat is complementary to the original circularized primer, an oligonucleotide probe complementary to the first primer sequence should hybridize to every repeat.

The significant difference between this amplification system and that of a cyclic amplification system is that replication of the circularized primer is continuous—there are no further denaturation steps, and the result is a long, single-stranded nucleic acid chain containing a number of repeats of the first primer sequence. In a cyclic amplification reaction, a denaturation step follows each replication step such that each new copy of the target sequence may serve as a template for the subsequent replication step. Thus, a cyclic amplification reaction is exponential in nature (given an unlimited supply of reagents) while a rolling circle amplification reaction is geometric in nature, since the newly synthesized repeats do not serve as templates for further replication.

A variant of the rolling circle amplification technique incorporates a third oligonucleotide primer which is complementary to the opposite strand of the first, circularized primer, such that the third primer is able to hybridize to the newly-synthesized repeats of the first, circularized primer. This permits the newly synthesized repeats to themselves serve as templates for replication, thereby increasing the amplification of the first circularized primer.

Any of the aforementioned amplification methods may be advantageously used to amplify a target moderately-repeated highly-conserved nucleic acid sequence for the purposes of identifying the presence or quantity of a selected chromosome or other nucleic acid molecule.

Detection of the Amplicon

The method by which the amplified target sequence may be detected depends on the detectable label utilized in the oligonucleotide probe or primer molecule. As described herein, these molecules may be conveniently tagged with a radioactive, fluorescent, colored, or chemiluminescent label. Detection of a radiolabel is generally performed by autoradiography or via a scintillation or gamma radiation apparatus. Detection of a fluorescent label may be accomplished by use of a fluorimeter. Colored or chemiluminescent labels are readily visible, but may be quantitatively detected through the use of a spectrophotometer.

Two types of information are available through the detection of the label associated with the primer or probe hybridized to the amplicon: the presence of the amplicon and/or the quantity of the amplicon. The presence of a detectable signal indicates the presence of the amplicon, since the label is preferably not detectable unless the probe or primer is either hybridized or not hybridized to the amplicon. This information is useful when it is important to know whether a particular nucleic acid molecule (e.g., a chromosome) is present in a cell (e.g., whether or not a cell possesses a Y chromosome). However, for other applications, it is important to be able to quantitate the number of a particular nucleic acid molecule (e.g., a chromosome) present in a cellular sample (e.g., to assess whether a trisomy is present, such as trisomy-21). Many of the labels discussed herein permit quantitative measurement, such as fluorophores, radiolabels, and calorimetric labels. By comparing the values obtained from measuring the label in a sample analyzed by the methodologies of the invention to that of a control sample having a known number of a selected nucleic acid molecule present, an increased or decreased number of this nucleic acid molecule in the sample from that of the control may be assessed.

As has been discussed, the preferred label of the invention is that which is only detectable when the oligonucleotide molecules to which it is attached are hybridized to their cognate sequences. The preferred label of the invention is detectable rapidly and without a requirement for additions to or modifications of the sample (e.g., without opening the reaction vessel), to permit real-time detection of the amplicon present without interfering with the progression of the amplification reaction, and further, to prevent contamination of the reaction or the surroundings.

Thus, in a preferred embodiment, the amplification reaction utilized in the methods is real-time PCR, and the detectable label of either the primer or the probe oligonucleotide molecule is a fluorophore, most preferably a molecular beacon.

IV. Methods for Monitoring of the Processes of the Invention

When performing an amplification reaction for a sequence specific to a selected nucleic acid molecule (e.g., a chromosome) in a few cells, a single cell or a portion thereof (e.g., an organelle), a small error in reagent concentration or protease activity may have a profound effect upon the outcome of the amplification reaction. Although the amplification of target sequences which are moderately-repeated and highly-conserved increases the overall number of available targets for amplification, these sequences are preferably all specific to one selected nucleic acid molecule, which may be present in only a few copies in the cell. If the target nucleic acid molecule is inaccessible, such as might occur if proteolysis of the cellular sample were incomplete, leaving intact nucleic acid binding proteins or proteins which aid in the folding and compaction of DNA, then a false negative result might be obtained. Further, if quantitative analysis of the number of the selected nucleic acid molecules present in the cell is being performed, then a skipped amplification initiation event or a lack of one or more amplification reagent components may result in an artificially low detectable amplicon signal Since the uses of the methods of the invention include applications such as preimplantation genetic diagnosis and forensic biology, the reliability of the result of the methods is critically important. Thus, the invention provides methods whereby both the reliability of initiation of the amplification reaction and the efficiency of the amplification reaction may be assessed.

In another embodiment of the invention, the quantity of the amplified moderately-repeated highly-conserved nucleic acid sequence detected at a first selected time (e.g., cycle) of amplification and the quantity of this amplified sequence detected at a later second selected time of amplification are compared to predetermined quantity values for the amplification of the same moderately-repeated highly-conserved sequence at these first and second selected times as an indication of the efficiency of the amplification reaction. These quantities are utilized not only as an indication of the presence or quantity of the selected nucleic acid molecule or portion thereof in the nucleic acid sample, but also as an indication of the reliability of initiation and efficiency of the amplification reaction. In a further embodiment, the quantity of the amplified nucleic acid sequence at three or more time points may be compared to equivalent time points in an appropriate control reaction.

Specifically, the signal detected from the labeled primer or probe either hybridized or unhybridized to the target moderately-repeated highly-conserved sequence is measured at a first selected time (e.g., cycle) of amplification. For convenience, this first selected time is typically the time at which the detectable signal first reaches a set threshold, such as ten times the standard deviation of the background noise of the detection system (for the detection of fluorescence in an ABI PRISM 7700 Sequence Detector (Applied BioSciences) the threshold value is conveniently set at 100 units), termed C_{T} (for cycle of threshold). The value obtained for the detectable signal at this first time (or the time at which C_{T} is achieved) is indicative of the reliability of initiation of the amplification reaction when compared to a standard control reaction utilizing the same amplification reagents and primer/probe molecules as the experimental reaction. The design of appropriate control reactions, both positive and negative, is well known in the art.

If C_{T} is at a significantly later time (e.g., cycle) of the reaction, or if the signal detected at the first selected time is significantly lower than that of the control reaction, then either the number of copies of the selected nucleic acid molecule in the sample is lower than that of the control reaction, or the reliability of initiation of the amplification reaction may be poor. These two options may be differentiated by comparison of C_{T} to a panel of control reactions having differing numbers of the selected nucleic acid molecule present in the reaction. Further, the sample may be re-tested utilizing oligonucleotide primer and probe molecules which specifically hybridize to a different sequence on the selected nucleic acid molecule to see if the C_{T} obtained is repetitive of the earlier data. If the C_{T} obtained from reactions utilizing two different sets of primers/probes repeatably yields a C_{T} which is equivalent to that of a control reaction in the panel having a particular number (e.g., 1 copy) of the selected nucleic acid molecule present (with the understanding that a separate panel of control reactions is required for each primer/probe combination), then it is likely that the experimental reaction has an equivalent number of the selected nucleic acid molecule present (e.g., 1 copy). If the C_{T} obtained from the experimental reactions fluctuates from sample to sample, or if the C_{T} obtained from experimental reactions utilizing one set of primer/probe molecules suggests (in comparison with a panel of control reactions) a number of the selected nucleic acid molecule present in the sample which is different that that obtained from experiments utilizing different primer/probe molecules specific for the same selected nucleic acid molecule, then it is likely that the reliability of the initiation of the amplification reaction is poor in the experimental samples.

In the instance where the reliability of initiation of the amplification reaction is poor, it will be apparent to the practitioner skilled in the art which experimental conditions may be modified to rectify the experimental error resulting in the low reliability of initiation of the reaction. Since reactions in which amplification is initially delayed will also have significantly lower overall levels of amplicon, it is preferred that such reactions are discarded from further analysis if the purpose is diagnostic in nature.

There are also experimental errors which may permit efficient initiation of an amplification reaction using the methods of the invention, but which result in premature stalling or slowing of the reaction. Such errors result in a decreased efficiency of amplification. These experimental errors may include the utilization of incorrect concentrations of amplification reagent components, such that one or more of the components is exhausted before the end of the overall amplification reaction. A similar effect may be seen if there is a malfunction with the apparatus performing the thermal cycling; if the appropriate temperatures for denaturation or annealing are not attained, for example, then the amplification reaction may not be able to progress. By detecting the quantity of amplicon at a later, second selected time (e.g., cycle) of amplification and comparing it to the quantity detected at the earlier, first selected time of amplification, a ratio is obtained which is indicative of the rate at which amplification of the target sequence is taking place in the time period between the first and second selected times. If the value obtained at the second selected time is not different or is only slightly different than that at the first selected time, then the amplification reaction has stalled. By comparing the obtained values (and their ratio) to those obtained in a standard control reaction utilizing the same amplification reagent and primer/probe molecules, it is possible to assess the efficiency of the amplification reaction in comparison with standard values. If the efficiency of amplification of the experimental sample is significantly different than that of the control reaction(s), then the sample should be discarded from further analysis. In such an instance, it will be apparent to the practitioner skilled in the art which experimental conditions may be modified to rectify the experimental error resulting in the low efficiency of amplification of the reaction.

Alternately, in another embodiment, the time at which the quantity of the detectable signal in the reaction reaches predetermined lower and higher quantity values may be assessed and compared to the times at which these values are reached in control reactions as an indication of the presence and/or quantity of a selected nucleic acid molecule in the sample and also as an indication of the reliability of initiation and efficiency of the amplification reaction. In a further embodiment, the times at which the detected amplified nucleic acid sequence reaches three or more predetermined quantity values may be assessed and compared to the times at which these values are attained in appropriate control reactions. In such embodiments, the methods of assessing the efficiency of the amplification reaction are similar to those described above, with the exception that a comparison of the time (e.g., cycle) at which a predetermined quantity value is reached is compared to the time at which it is attained in an appropriate control reaction, rather than the previously discussed comparison of the quantities of the amplicon detected at predetermined times of the amplification reaction.

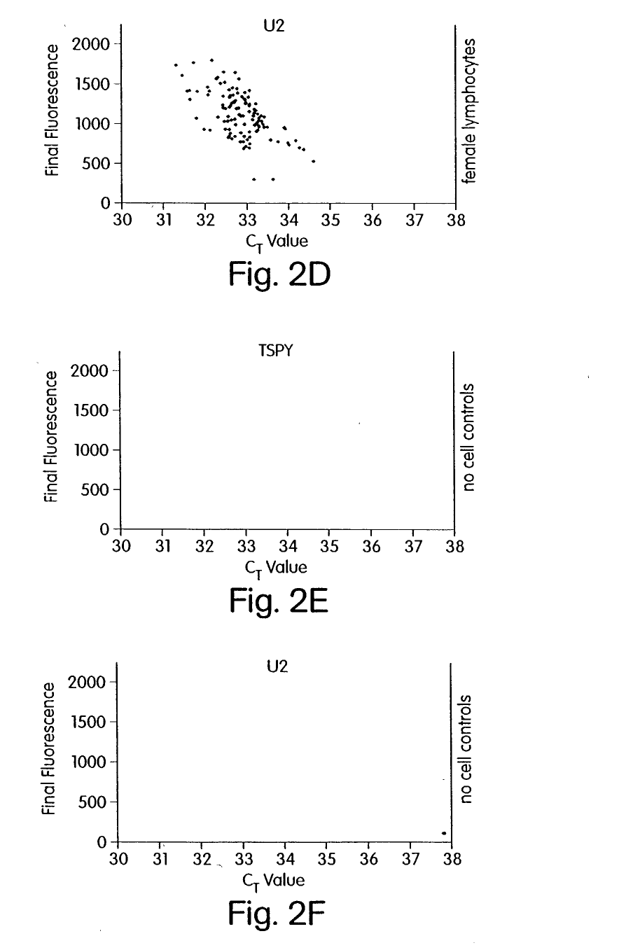

In another embodiment, the invention provides cutoff values for the quantity of amplicon detected at a first selected time (e.g., cycle) of amplification and at a second selected time of amplification, and for the ratio of these two values such that erroneous reactions may be readily discarded from the analysis. Such cutoff values may be conveniently presented in the format of a graph (for example, of the value at C_{T} versus the fluorescence detected at a second later selected time), in which unacceptable values, when also plotted on the graph, fall outside of an indicated boundary (see, e.g., FIG. 3).

In another embodiment, to address the problem of potential false-negative results, wherein the selected nucleic acid molecule (e.g., a chromosome) may not be available for hybridization due to inefficient or incomplete proteolysis, or wherein one or more errors in the amplification reagent fail to permit initiation of amplification, as discussed above, the invention provides a method whereby an internal control amplification reaction is included in the cell sample analysis. For example, employing the methods of the invention, it is possible to amplify two different moderately-repeated highly-conserved sequences simultaneously. It is highly unlikely that two negative results (e.g., lack of detectable amplicon product) should result (e.g., while the Y chromosome may legitimately be lacking in the cellular sample, chromosome 17 should not be lacking). If a doubly negative result is achieved, then it is likely that the results represent false-negatives and that there is a source of error in the reaction which requires correction. If the internal control moderately-repeated highly-conserved sequence amplifies appropriately, then the negative result for the selected nucleic acid molecule is likely real (with the caveat that the primers specific for the moderately-repeated highly-conserved sequence must be verified to result in a positive signal in a sample known to contain the selected nucleic acid molecule).

Alternately, in another embodiment, an end-point measurement may be taken for an experimental reaction, either in the form of the time of the reaction at which an increase in the amount of label is no longer observed (e.g., a finished reaction), or the maximum detected value for the label. This end-point measurement may conveniently be compared to those of standard control reactions utilizing similar reaction conditions and the same oligonucleotide primer and probe molecules for an indication of the presence and/or quantity of a selected nucleic acid molecule present in the sample.

Further, it will be appreciated by one skilled in the art that this methodology may be used to screen amplification reagents or primer/probe molecules to ensure that these reagents function properly when included in the methods of the invention.

V. Kits for the Practice of the Methods of the Invention

The invention provides kits for the convenient practice of the methods of the invention. In one embodiment, the invention provides a kit for the preparation of accessible nucleic acid molecules from a cell, a group of cells, or a portion of a cell, containing a protease-based lysis buffer comprising an ionic detergent, a protease, and a buffering agent in at least one container. In a preferred embodiment, the lysis buffer does not contain compounds which are inhibitory to the action of the protease and/or are inhibitory to a subsequent amplification step. In another preferred embodiment, the kit further contains instructional materials and/or equipment useful for the implementation of the kit (e.g., pipets, microfuge tubes, etc.). In a particularly preferred embodiment, the protease is proteinase K, the ionic buffer is SDS, and the buffering agent is Tris-HCl, and the buffer lacks Mg²⁺ and chaotropic salts.

In another embodiment, the aforementioned kit further comprises at least two oligonucleotide primer molecules sufficiently complementary to a target moderately-repeated highly-conserved nucleic acid sequence of a selected nucleic acid molecule such that they may serve as amplification primers for the amplification of a plurality of the copies of the target moderately-repeated highly-conserved nucleic acid sequence present in the sample. In a preferred embodiment, one or more of the oligonucleotide primer molecules may be detectably labeled. In a particularly preferred embodiment, this label is detectable only when the primer is either hybridized or not hybridized to the target moderately-repeated highly-conserved nucleic acid sequence for which it is specific.

In another embodiment, the invention provides a kit for the detection and/or quantification of a selected nucleic acid molecule in a cell, a group of cells, or a portion of a cell, containing a protease-based lysis buffer comprising an ionic detergent, a protease, and a buffering agent in at least one container, at least a second container including two oligonucleotide primer molecules sufficiently complementary to a target moderately-repeated highly-conserved nucleic acid sequence of a selected nucleic acid molecule such that they may serve as amplification primers for the amplification of the plurality of the copies of the target moderately-repeated highly-conserved nucleic acid sequence present in the sample, and an oligonucleotide probe which is sufficiently complementary to the target moderately-repeated highly-conserved nucleic acid sequence such that it hybridizes to a plurality of the copies of the sequence present in the sample. In a preferred embodiment, the lysis buffer does not contain compounds that are inhibitory to the action of the protease and/or are inhibitory to a subsequent amplification step. In another preferred embodiment, the oligonucleotide probe may be detectably labeled. In a particularly preferred embodiment, this label is detectable only when the probe is either hybridized or not hybridized to the target nucleic acid sequence for which it is specific (e.g., a molecular beacon probe molecule). In a further preferred embodiment, the kit further contains instructional materials and/or equipment useful for the implementation of the kit (e.g., pipets, microfuge tubes, etc.). In a particularly preferred embodiment, the protease is proteinase K, the ionic buffer is SDS, and the buffering agent is Tris-HCl, and the buffer lacks Mg²⁺ and chaotropic salts. In yet another preferred embodiment, this kit may also include an amplification reagent in at least a third container, including a polymerase, a buffering agent, one or more salts, and deoxynucleotide triphosphate molecules.

In another embodiment, the invention provides a kit for the detection and/or quantification of a selected nucleic acid molecule in a sample containing nucleic acid molecules, including at least a first container at least two oligonucleotide primer molecules sufficiently complementary to a target moderately-repeated highly-conserved nucleic acid sequence of a selected nucleic acid molecule such that they may serve as amplification primers for the amplification of the plurality of the copies of the target moderately-repeated highly-conserved nucleic acid sequence present in the sample, and an oligonucleotide probe which is sufficiently complementary to the target moderately-repeated highly-conserved nucleic acid sequence such that it hybridizes to a plurality of the copies of the target moderately-repeated highly-conserved sequence present in the sample. In a preferred embodiment, either one or more of the primer molecules or the probe is detectably labeled. In a particularly preferred embodiment, the label is detectable only when the primer or probe with which the label is associated is either hybridized or not hybridized to the nucleic acid sequence to which it is specific (e.g., the primer or probe is a molecular beacon primer or probe molecule). In another preferred embodiment, the kit further includes in at least a second container an amplification reagent including a polymerase, a buffering agent, one or more salts, and deoxynucleotide triphosphate molecules. In another preferred embodiment, the kit further contains instructional materials and/or equipment useful for the implementation of the kit (e.g., pipets, microfuge tubes, etc.).

In another embodiment, the invention provides a kit for the detection and/or quantification of two or more selected nucleic acid molecules in a sample containing nucleic acid molecules. This kit includes, in at least a first container, a panel of oligonucleotide primer molecules containing at least two primer molecules specific for each selected nucleic acid molecule to be detected, wherein these primer molecules are sufficiently complementary to a target moderately-repeated highly-conserved nucleic acid sequence of the selected nucleic acid molecule such that they may serve as amplification primers for the amplification of the plurality of the copies of the target moderately-repeated highly-conserved nucleic acid sequence present in the sample. The kit also contains, in at least a second container, a panel of oligonucleotide probe molecules containing at least one probe specific for each selected nucleic acid molecule to be detected, wherein these probe molecules are sufficiently complementary to the target moderately-repeated highly-conserved nucleic acid sequence such that they hybridize to a plurality of the copies of the target moderately-repeated highly-conserved sequence present in the sample. In a preferred embodiment, either one or more of the primer molecules or the probe is detectably labeled. In a particularly preferred embodiment, the label is detectable only when the primer or probe with which the label is associated is either hybridized or not hybridized to the nucleic acid sequence to which it is specific (e.g., the primer or probe is a molecular beacon primer or probe molecule). In another preferred embodiment, the kit further includes in at least a second container an amplification reagent including a polymerase, a buffering agent, one or more salts, and deoxynucleotide triphosphate molecules. In another preferred embodiment, the kit further contains instructional materials and/or equipment useful for the implementation of the kit (e.g., pipets, microfuge tubes, etc.).

In another embodiment, the invention provides a kit for the detection and/or quantification of two or more selected nucleic acid molecules in a single cell, a group of cells, or a portion of a cell (e.g., an organelle). This kit includes, in at least a first container, a protease-based lysis buffer comprising an ionic detergent, a protease, and a buffering agent. The kit further includes, in at least a second container, a panel of oligonucleotide primer molecules containing at least two primer molecules specific for each selected nucleic acid molecule to be detected, wherein these primer molecules are sufficiently complementary to a target moderately-repeated highly-conserved nucleic acid sequence of the selected nucleic acid molecule such that they may serve as amplification primers for the amplification of the plurality of the copies of the target moderately-repeated highly-conserved nucleic acid sequence present in the sample. The kit also includes, in at least a third container, a panel of oligonucleotide probe molecules containing at least one probe specific for each selected nucleic acid molecule to be detected, wherein these probe molecules are sufficiently complementary to the target moderately-repeated highly-conserved nucleic acid sequence such that they hybridize to a plurality of the copies of the target moderately-repeated highly-conserved sequence present in the sample. In a preferred embodiment, either one or more of the primer molecules or the probe is detectably labeled. In a particularly preferred embodiment, the label is detectable only when the primer or probe with which the label is associated is either hybridized or not hybridized to the nucleic acid sequence to which it is specific (e.g., the primer or probe is a molecular beacon primer or probe molecule). In a preferred embodiment, the lysis buffer does not contain compounds which are inhibitory to the action of the protease and/or are inhibitory to a subsequent amplification step. In a particularly preferred embodiment, the protease is proteinase K, the ionic buffer is SDS, and the buffering agent is Tris-HCl, and the buffer lacks Mg²⁺ and chaotropic salts. In another preferred embodiment, the kit further includes in at least a second container an amplification reagent including a polymerase, a buffering agent, one or more salts, and deoxynucleotide triphosphate molecules. In another preferred embodiment, the kit further contains instructional materials and/or equipment useful for the implementation of the kit (e.g., pipets, microfuge tubes, etc.).

In another embodiment, one or more of the kits of the invention is specific for the detection of human chromosome 17 in a human cell, and contains primers which specifically hybridize to a target moderately-repeated highly-conserved sequence (e.g., the U2 sequence) of human chromosome 17. Such a kit may additionally comprise an oligonucleotide probe that specifically hybridizes to a plurality of the copies of the target sequence of human chromosome 17.

In another embodiment, one or more of the kits of the invention is specific for the detection of the human Y chromosome in a human cell, and contains primers which specifically hybridize to a target moderately-repeated highly-conserved sequence (e.g., the TSPY sequence) of the human Y chromosome. Such a kit may additionally comprise an oligonucleotide probe that specifically hybridizes to the plurality of the copies of the target sequence of the human Y chromosome.

VI. Uses of the Invention

The compositions and methods of the invention permit the analysis of the nucleic acid content of groups of cells, single cells, and a portion of a cell (e.g., an organelle) and as such are applicable to a variety of uses, including research, forensic science and diagnostic applications.

Diagnostic Applications

The compositions, methods, and kits of the invention are also useful in a variety of diagnostic applications, such as preimplantation genetic diagnosis (PGD). In PGD, embryos may be tested to establish either sex or the presence of nonstandard numbers of one or more chromosomes (e.g., trisomy). In situations in which the mother carries a recessive X-linked genetic disease, such as Duchene muscular dystrophy or hemophilia, on one of her two X chromosomes, any son born to the woman will have a 50% chance of being affected by the disease. Similarly, sons born to males having an X-linked genetic disease will be free of the disease, while all of the daughters will be either carriers of the disease or will be affected by the disease. Sons born to a male having a Y-linked genetic disease or condition will be similarly affected, whereas daughters will not be affected. Thus, in many cases it may be of advantage to select an embryo for implantation of the appropriate sex such that genetic diseases may be avoided.

The compositions and methods of the invention permit the genomic analysis of embryos prior to implantation to assess whether all chromosomes are present in the correct number of copies. Devastating genetic diseases and conditions have been linked to the presence of too many or two few copies of a particular chromosome, such as trisomy-18 (Edward's syndrome), trisomy-13 (Patau's syndrome), trisomy-21 (Down's syndrome), an additional X chromosome (e.g., XXY (Klinefelter's syndrome)), a single X chromosome (Turner's syndrome), and trisomy-X (triple-X syndrome). By permitting the selection of embryos not having such chromosomal abnormalities, it is possible to avoid the presence of such genetic diseases. Further, it is possible to utilize the methods of the invention to analyze the samples taken during amniocentesis for genetic abnormalities.

The methods and compositions of the invention are also useful for the detection of certain types of non-genetic diseases. For example, many viruses function by incorporating their nucleic acid molecule into that of the host cell, in which it may lie dormant until a specific event triggers viral production. It is possible to use the methods of the invention to detect the presence of a viral nucleic acid molecule (e.g., HIV or hepatitis) within the genetic complement of, for example, a human cell through the amplification and detection of moderately-repeated highly-conserved sequences specific to the suspected viral nucleic acid molecule in samples of cells from a subject being tested. The sensitivity of the assay methods of the invention is such that even a single copy of a viral nucleic acid molecule present in the cell (such as in the case when the virus is dormant) should be readily detectable.

Similarly, the methods of the invention may be utilized to detect the presence of foreign cells in a subject. For example, the presence of bacteria in various bodily fluids or tissues (e.g., blood, urine, or spinal fluid) can be readily detected by the amplification of sequences (e.g., moderately-repeated highly-conserved sequences) specific to the bacterial chromosome(s). This is particularly useful for the identification of an infection by a pathogen that is otherwise difficult to detect, such as Borrelia burgdorferi (the causative agent of Lyme disease, which is largely sequestered in synovial fluid), and bacteria which multiply and disseminate inside of host cells without exposure to the bloodstream (e.g., Salmonella or Shigella). The presence of fetal cells in the mother's blood and cells of the mother in the blood of the fetus may also be detected. Since certain diseases (e.g., scleroderma) have been associated with the presence of fetal cells in the mother's blood, this may be relevant to the early detection and/or prevention of diseases related to this accidental exchange of cells.

The methods and compositions of the invention may be applied to the detection of cancerous cells. For example, it is possible to detect specific chromosome rearrangements (e.g., translocations) or changes in gene expression (e.g., by detecting the number of one or more selected mRNA molecules) in a single cell or groups of cells isolated from tumor masses.

Further, it is possible, using the methods of the invention, to discriminate between two different alleles having a single base-pair mismatch, due to the fact that the hybridization steps are performed under high stringency. Certain oncogenes linked to the transformation of normal cells to a cancerous state are due to only a single base-pair mismatch (e.g., a single base-pair alteration in codon 12 of the ras gene converts the gene to an oncogene). The methods of the invention, then, may be used to identify the presence of a known oncogene. By screening cells from a subject with panels of primers specific for different oncogenes, it may be possible to assess the risk of development of certain kinds of cancers in the subject. Further, it is possible to screen cancerous cells from a subject in order to identify any oncogenes which may have contributed to the development of the tumor, which is useful not only for the identification of new oncogenes, but also for identifying the origin of the tumor, such that treatment may be tailored appropriately. This is particularly useful in the treatment of cancers by gene therapy. These applications of the methods of the invention are particularly suited for cancer detection/diagnosis, since these diagnoses may be performed on single cells, and thus permit a number of analyses from even a minute tumor or a biopsy sample, such that even very early-stage cancers may be diagnosed and identified.

While such genetic analyses have been previously available, through techniques such as fluorescence in-situ hybridization (FISH) and various PCR methodologies, the methods of the invention offer a substantial improvement over the methods previously utilized. When examining an embryo, an amniocentesis sample, or tissues that are difficult, painful, or deleterious to extract (e.g., nerve cells, spinal fluid, or bone marrow), the preferred sample size is very small; the methods of the invention permit a genetic analysis of each single cell or portion thereof (e.g., a nucleus) in the sample, such that even a small sample size may yield multiple analyses, giving statistically significant results. Further, the methods of the invention also include quality control steps, such that reactions that do not meet specific criteria may be discarded when the purpose is diagnostic in nature. Thus, not only can multiple analyses be performed on a very small biological sample, but reactions which have failed to meet reliability criteria may be discarded from the overall analysis of the sample (when the purpose is diagnostic in nature), ensuring that the data generated is reliable.

Forensic Applications

Forensic science is concerned with the scientific analysis of evidence from a crime. Forensic biology applies the experimental techniques of molecular biology, biochemistry and genetics to the examination of biological evidence for the purpose, for example, of positively identifying the perpetrator of a crime. Typically, the sample size of such biological evidence (e.g. hair, skin, blood, saliva, or semen) is very small. The methods of the invention permit the detection of particular chromosomes from a single cell or portion thereof, making it possible to identify, for example, the sex or species of origin of even minute biological samples. Panels of probes specific for different moderately-repeated highly-conserved sequences characteristic for different chromosomes or species can be used to identify a given tissue by species and/or by sex.

In a similar fashion, the compositions and methods of the invention, (e.g., oligonucleotide primer or probe molecules specific for different moderately-repeated highly-conserved sequences which are characteristic of chromosomes from different species of organisms), can be used to screen samples or tissue culture for contamination (for example, to screen for the presence of bacterial cells in a culture of human cells) which may interfere with the correct analysis of the biological sample.

Research Applications

The methods and compositions of the invention have a variety of research applications. First, they are useful for any research application in which genetic analyses must be performed on very small numbers of cells, such as in conjunction with cell-sorting techniques that result in the selection of few cells (e.g., laser catapulting, or fluorescence-assisted cell sorting (FACS)). Second, the methods of the invention provide a simple method for detecting the presence of an introduced mutation in a cell, particularly a knockout mutation. Further, the invention permits an examination of the relative rate of multiplication of different nucleic acid sequences in a cell, (in that it is possible to discriminate between a single copy of a gene or chromosome and multiple copies of that gene or chromosome) and may enable researchers to detect the order of replication of sequences in a given cell. This information, in turn, may yield useful information about those sequences that are most important to the functioning of the cell, or those sequences that are necessary for further cellular replication. Other applications of the compositions and methods of the invention for research uses will be readily apparent to those skilled in the art.

This invention is further illustrated by the following examples, which should not be construed as limiting. The contents of all references, patents, and published patent applications cited throughout this application are incorporated herein by reference.

### EXAMPLES

Lymphocytes were chosen for experimental analysis because they serve as good examples of nondividing cells, thus preventing cell division after isolation of the cell but prior to utilization in a method of the invention. These immune cells are also known to be involved in a number of genetic diseases (e.g., leukemia), and therefore the ability to detect genetic alterations or abnormalities in these cells may be of therapeutic or diagnostic utility.

### Example 1

### Preparation and Handling of Lymphocytes

Blood from single male and female donors was drawn directly into tubes containing EDTA to prevent clotting. Three milliliters (ml) of whole blood was layered over 3 ml of Histopaque-1077 (Sigma, St. Louis, Mo.) and centrifuged at 400× g for 30 min. Most of the plasma was discarded and the layer of mononuclear leukocytes (predominantly lymphocytes) was collected and washed 3 times with DPBS lacking calcium or magnesium (PBS, Sigma, cat. no. D-8537). The cells were resuspended in 70% PBS, 30% glycerol and chilled on ice. Aliquots were placed in screw-cap, 0.5 ml centrifuge tubes, and frozen in liquid nitrogen.

For the transfer of individual lymphocytes, an aliquot of the cell suspension was thawed, and 1 μl was added to 3 ml of PBS in a Costar ultra-low-attachment culture plate (Fisher Scientific, Pittsburgh, Pa., cat. no. 07-200-601). A single cell was aspirated into a finely-drawn glass pipette while viewing at 100× magnification with an Olympus IX70 microscope. The pipette contents were expelled directly into 10 μl of protease-based lysis buffer (see below) in a 0.2 ml MicroAmp optical PCR tube (PE Applied Biosystems, Foster City, Calif.). The tube was kept on ice until the transfer of all cells was complete.

### Example 2

### Preparation and Handling of Blastomeres

Non-viable embryos deemed unsuitable for transfer to patients were obtained for experimental analysis following written patient consent and Internal Review Board approval at the Institute for Reproductive Medicine and Science of Saint Barnabas. Embryos on day 3 or day 4 post-insemination (4 to 12 cell stage) were treated briefly in acidified Tyrode's solution to remove the zona pellucida, then rinsed 3 to 5 times in PBS containing 0.1% polyvinylpyrrolidone (PVP-40, Sigma) and incubated approximately 30 min. in that solution. Embryos were disaggregated into individual blastomeres by repeated aspiration into a narrow diameter plastic pipet with a bore size of 0.16 mm (Drummond Scientific Company). A small number of blastomeres were obtained by biopsy, rather than disaggregation. In all cases, each blastomere was rinsed twice in PBS containing 0.1% PVP-40, once in PBS containing 0.01% PVP-40, then transferred directly into 10111 protease-based lysis buffer (see below) in a 0.2 ml MicroAmp optical PCR tube. Control samples were prepared by transferring a similar volume of final wash buffer (<1 μl) to the lysis buffer. All samples were kept on ice until transfer of all samples was complete.

### Example 3

### Cell Lysis and Nucleic Acid Preparation

Protease-Based Lysis Buffer

The lysis buffer utilized in the preparation of cellular nucleic acids consisted of 100 μg/ml proteinase K (Fisher Scientific), 5 μM SDS, and 5 mM Tris, pH 8.3 at 25° C. (Trizma™ pre-set crystals, Sigma). The buffer was kept on ice and aliquotted into volumes sufficient for single experiments, then stored at −20° C.

Cellular Lysis and Protein Digestion

After thawing, 10 μl of lysis buffer was pipetted into each optical PCR tube and kept on ice. Following the above-described transfer of single lymphocytes to each tube, samples were placed in a thermal cycler block preheated to 50° C. A heated cover was positioned over the samples to prevent condensation, and the samples were incubated at 50° C. for 60 min. A subsequent incubation was performed at 95° C. for 10 min. to inactivate the proteinase K.

### Example 4

### Amplification

Oligonucleotide Primer and Probe Molecules

Primer and probe molecules were designed with the aid of Oligo 5.0 software (National Biosciences, Inc., Plymouth, Minn.). Desalted primers of the selected sequences were purchased from Life Technologies (Gaithersburg, Md.). Theoretical folding structures of the amplified sequences as determined by oligonucleotide nearest-neighbor thermodynamics (SantaLucia (1998) Proc. Natl. Acad. Sci. USA 95:1460-1465) were examined by submitting the sequence for analysis at the following internet site: http://mfold.wustl.edu/˜folder/dna/form1.cgi (moving to http://www.rpi.edu/˜zukerm in the fall of 2000). Oligonucleotide probes utilizing molecular beacon technology as a detectable label were designed according to the methods of Tyagi, S. and Kramer, F. R. (1996) as detailed on the internet site: http://molecular-beacons.org. The guidelines included the following parameters: 1) Amplicon regions that could form stable hairpins were avoided as possible targets, as were sequences with strong complementarity with any of the primers. 2) The Tₘ of the hybridized loop sequence was 5 to 10° C. higher than the Tₘ of the primers. 3) The oligonucleotide folding program predicted a hairpin as the only stable structure for the oligonucleotide probe in the absence of target at the PCR annealing temperature. The predicted Tₘ for that hairpin structure was about 10° C. above the annealing temperature. Oligonucleotide probes detectably labeled with the molecular beacon technology were purchased from Research Genetics, Inc. (Huntsville, Ala.).

For TSPY amplification, the sense primer sequence was 5′ ATACAGGGCTTCTCATTCCA 3′ (SEQ ID NO: 4) and the antisense primer sequence was 5′ GTTAGATCCTGCGAAGTTGTG 3′ (SEQ ID NO: 5). These primers amplify a 133 bp segment of TSPY exon 4 and were based on sequences from clone Y-231 (Zhang, J. S. et al. (1992) Hum. Mol. Genet. 1:717-726). The TSPY probe sequence was 5′ CGCGCTTTGTGGTGTCTGCGGCGATAGGCAGCGCG 3′ (SEQ ID NO: 6) with the fluorophore TET covalently attached to the 5′ end and the quencher 4-(4-dimethylaminophenyl azo)benzoic acid (DABCYL) covalently attached to the 3′ end.

Amplification of the U2 small nuclear RNA gene (Pavelitz, T. et al. (1995) EMBO J 14:169-177) generated a 175 bp sequence with sense primer, 5′ AAGAAATCAGCCCGAGAGT 3′ (SEQ ID NO: 1), and antisense primer, 5′ CTTGATCTTAGCCAAAAGGT 3′ (SEQ ID NO: 2). The antisense primer contains a mismatch to its target at the 3′ end in order to reduce possible dimerization with the TSPY primers, and to reduce the efficiency of priming such that the TSPY amplification is preferentially amplified under competitive conditions. The U2 molecular beacon sequence was 5′ CTGGCCTGTCTCGTCCACAGCGCTATTGAGGCCAG 3′ (SEQ ID NO: 3) with the fluorophore FAM covalently attached to the 5′ end and the quencher DABCYL covalently attached to the 3′ end. Electrophoresis through a 3% agarose gel was used following PCR with separate or multiplexed primer pairs to confirm the production of amplicons with the expected sizes for U2 and TSPY.

Amplification

Fifteen microliters of concentrated polymerase chain reaction (PCR) reagent mixture were added to each tube containing either one or two lysed cells (or to control tubes not containing a cell) yielding final concentrations of 50 mM Tris, pH 8.3 (at 25° C.), 3.5 mM MgCl_{2, 0.4} mM of each dNTP, 0.3 μM of each primer, 0.3 μM of each probe molecule, and 0.5 units Taq polymerase (Promega, Madison, Wis.) per 25 μl reaction. Taq polymerase was preincubated with TaqStart antibody (Clontech, Palo Alto, Calif.) for 5 minutes at room temperature before it was added to the PCR mixture to inhibit polymerase activity until the first denaturation step (hotstart PCR).

Amplification and fluorescence detection was carried out using an ABI Prism 7700 Sequence Detector (PE Applied Biosystems). The cycling profile included an initial denaturation step at 95° C. for 3 min, followed by 38 cycles at 95° C. for 10 sec, 58° C. for 45 sec, and 72° C. for 10 sec, with fluorescence readings taken during the 58° C. step.

Contamination Control

When working with a small starting sample of nucleic acids which are to be amplified, contamination must be kept to a minimum. Therefore, preparation of the lysis buffer and the PCR reagent mixture was carried out in a room restricted to those activities, utilizing dedicated pipetters and supplies. All pipetting was done within PCR enclosure hoods (Labconco or similar with plexiglass front panels) using aerosol-resistant pipet tips. Hood surfaces, pipetters, and supplies were treated with UV light between uses and were touched only with gloved hands. Surfaces were treated approximately once per week with 10% chlorine bleach. Investigators wore disposable surgical masks and caps, gloves with extended cuffs, and lab coats that remained in the PCR preparation room.

Single lymphocytes were aspirated while viewing through an inverted microscope on the open bench and were then expelled into sample tubes opened in an adjacent PCR enclosure. Each tube was recapped immediately. All manipulations of embryos and blastomeres were carried out within a laminar flow hood. Following the lysis incubation, samples were returned to the PCR enclosure, in which PCR reagent mixture was added to the tubes, and the tubes were resealed with new caps.

Following PCR amplification, sample tubes were either sealed within a bag for disposal or taken to a separate lab for electrophoretic analysis. Electrophoretic equipment and supplies were never brought into the PCR laboratories. Investigators wore disposable lab coats when handling PCR products and were not permitted to participate in PCR reagent preparations or to perform PCR amplification reactions later on the same day.

### Example 5

### Statistical Analysis

The utility and efficiency values obtained for blastomeres from embryos with different levels of fragmentation were compared using two-sample contingency tests for homogeneity of binomial proportions. Blastomere concordance values were compared using Fisher's exact test.

### Example 6

### Discrimination Between One and Two Cells Based on Chromosomal Content

Fifteen microliters of concentrated polymerase chain reaction (PCR) reagent mixture were added to each tube containing either one or two lysed cells (or to control tubes not containing a cell), yielding final concentrations of 100 mM Tris, pH 8.3 (at 25° C.), 3.5 mM MgCl₂, 0.4 mM of each dNTP, 0.3 μM of each primer (the sense primer utilized for this experiment was SRY2232 left, 5′ AAAGGCAACGTCCAGGATAG 3′ (SEQ ID NO: 7), and the antisense primer was SRY2480 right, 5′ AATTCTTCGGCAGCATCTTC 3′ (SEQ ID NO: 8), 0.3 μM of a probe detectably labeled with Sybergreen (FMC corporation), and 1.0 units Taq polymerase (Promega, Madison, Wis.) per 25 μl reaction. Taq polymerase was preincubated with TaqStart antibody (Clonetech, Palo Alto, Calif.) for 5 minutes at room temperature before it was added to the PCR mixture to inhibit polymerase activity until the first denaturation step (hotstart PCR).

Amplification and fluorescence detection was carried out using an ABI Prism 7700 Sequence Detector (PE Applied Biosystems). The cycling profile included an initial denaturation step at 95° C. for 3 min, followed by 45 cycles at 95° C. for 10 sec, 55° C. for 10 sec, 72° C. for 5 sec, and 83° C. for 5 sec, with fluorescence readings taken during the 83° C. step.

The results show that the two cell samples demonstrated higher fluorescence readings than single cell samples, with the two cell signal being approximately two-fold higher than that of the one cell signal over at least the four cycles following the cycle of threshold (C_{T}). Since the primers utilized in this experiment hybridize specifically to the moderately-repeated highly-conserved gene TSPY on the Y chromosome, this experiment demonstrates the ability of the methods of the invention to discriminate between a single copy of a chromosome (the single copy of the Y chromosome in one cell) and two copies of the chromosome (the single copy of the Y chromosome in each of two cells) in a reaction.

### Example 7

### Optimization of the Assay

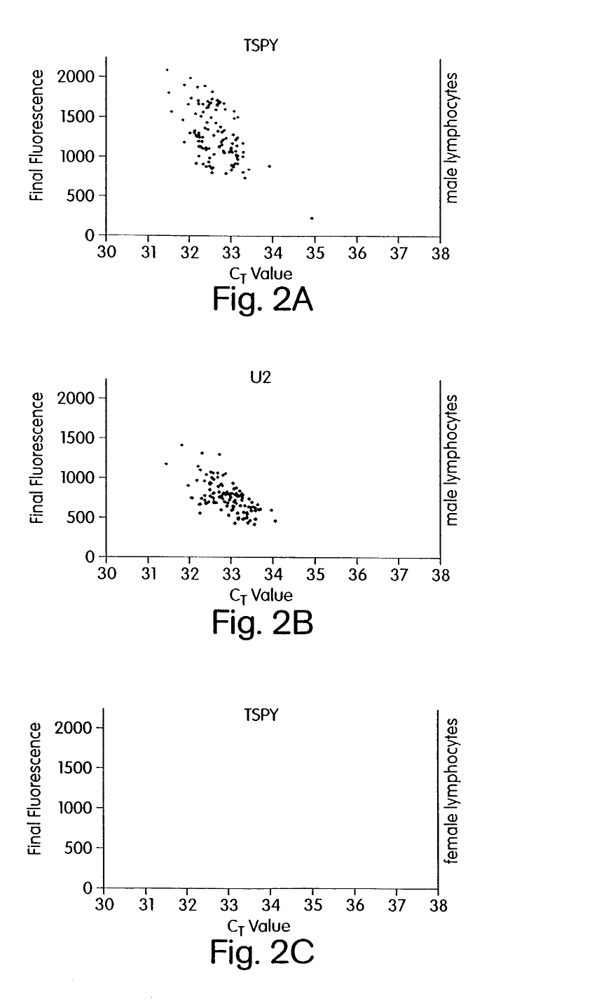

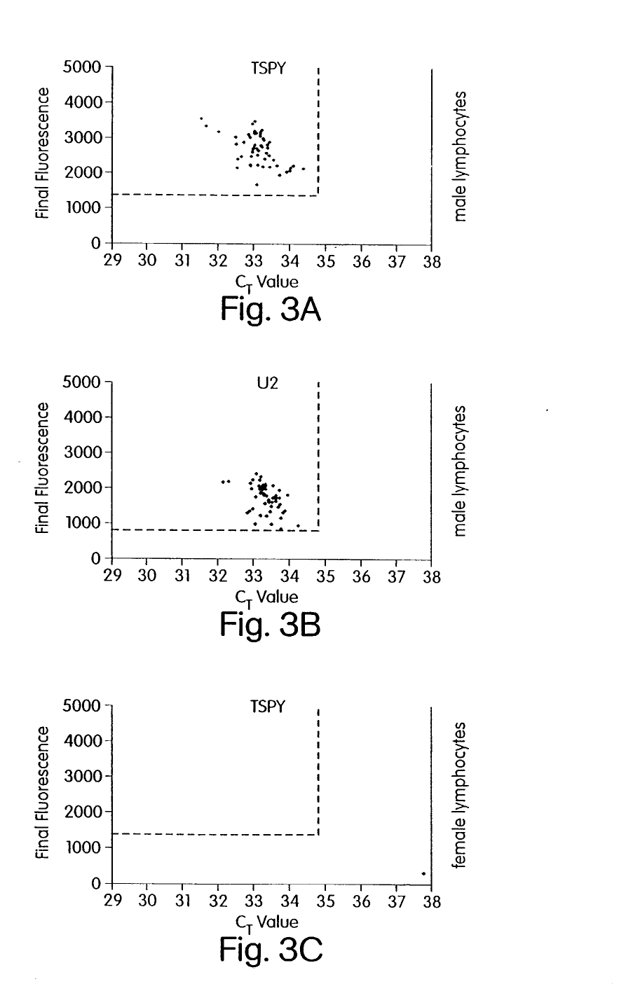

240 reactions were prepared using single male (120 reactions) or female (120 reactions) lymphocytes to determine the reliability of the assay. Among the 240 reactions, 8 (3.3%) showed no U2 or TSPY signals at all, probably because cells were not successfully transferred into those reaction tubes. Of the 232 reactions that did have signals, all had at least one with a C_{T} value of less than 35. A total of 114 of those reactions contained a male lymphocyte, and 113 generated a TSPY signal (FIG. 2A). The single remaining sample lacked a TSPY signal, but did generate a strong U2 signal. The samples with TSPY signals also had U2 signals with similar C_{T} values (FIG. 2B), except for one sample with the lowest TSPY fluorescence intensity that lacked a U2 signal. As expected, all of the 118 female cells reactions that generated a U2 signal lacked a TSPY signal (FIGS. 2C and 2D). The mean and standard deviation for the C_{T} and final fluorescence values for all reactions with signals are presented in FIG. 4 (first experimental series). An additional set of 72 control reactions was also tested to screen for possible contamination within the laboratory. No TSPY signals were observed in any of these reactions, while 7 reactions exhibited U2 signals with C_{T} values >36 and fluorescence intensities far weaker than any of the robust signals observed in reactions containing a lymphocyte (FIGS. 2E and 2F).

Despite the high percentages of robust reactions in FIG. 2, a close examination of the data suggested that PCR conditions were not yet fully optimized. In particular, comparison of the data in FIGS. 2B and 2D reveals that the average U2 signal obtained from male lymphocytes had lower final fluorescence intensity than that obtained from female lymphocytes, although the mean C_{T} values of the two data sets are comparable (FIG. 4, first experimental series). The observation suggests that simultaneous amplification of TSPY in the male samples might partially inhibit U2 amplification during the final few PCR cycles. In contrast, amplification of U2 did not suppress TSPY amplification, even when a single male cell was tested in the presence of 100 female genomes. In order to minimize effects of TSPY on U2, the concentrations of Taq polymerase and Tris buffer were increased. This adjustment increased the final fluorescence intensity of all signals and brought the U2 fluorescence in male cell samples closer to that in female cell samples (FIG. 4, second experimental series, and FIGS. 3B and 3D).

### Example 9

### Optimized Gene Detection and Gender Diagnosis in Lymphocytes

In order to establish objective quantitative criteria for diagnosing the presence of the Y chromosome in single human cells, 54 samples of single male lymphocytes and 54 samples of single female lymphocytes were tested under the fully-optimized conditions in parallel with blastomere samples (see below), and the results were plotted in terms of C_{T} value and final fluorescence (FIGS. 3A-3D). One male lymphocyte sample showed no TSPY or U2 signal, presumably because the cell was not successfully transferred into that reaction tube. The C_{T} and final fluorescence values for the remaining 107 lymphocyte samples (FIG. 4, second experimental series) were used to define a robust reaction as one that yields a C_{T} value not greater than 3 standard deviations above the mean and final fluorescence not less than 3 standard deviations below the mean. Thus, a robust TSPY signal has a C_{T} of less than 34.7 and final fluorescence of at least 1349 units, and a robust U2 signal in the absence of a TSPY signal has a C_{T} of less than 34.5 and final fluorescence of at least 811 units. These limits are indicated by the dashed lines in FIG. 3. All 53 signal-positive male lymphocyte samples yielded both robust TSPY and robust U2 signals. All 54 female lymphocytes yielded robust TSPY signals for only U2. Two female lymphocyte samples showed low-level fluorescence for TSPY in the final cycles (C_{T}>37), which was easily distinguished from robust TSPY signals in samples of male lymphocytes. None of 16 control samples without a lymphocyte yielded either signal.

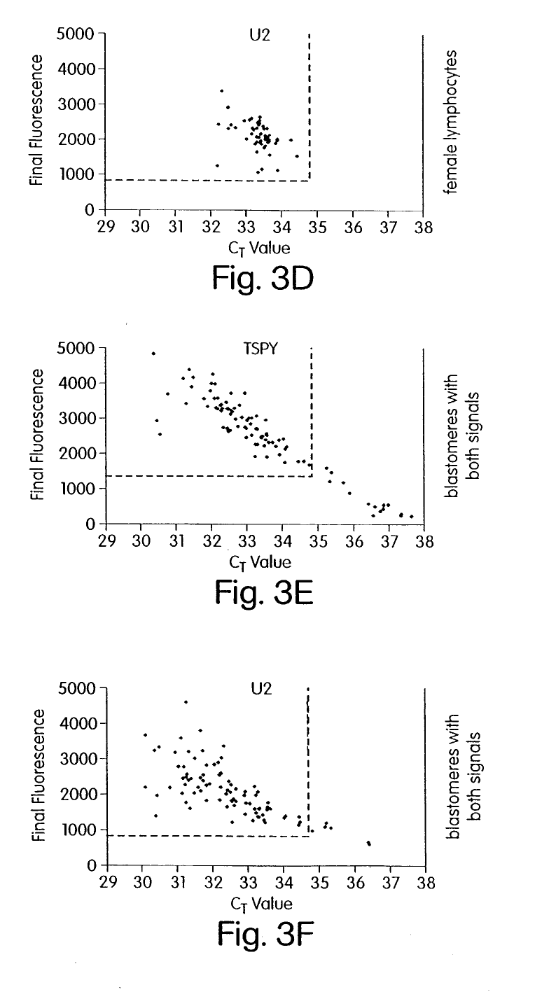

FIG. 5 summarizes the lymphocyte data from the diagnostic perspective. The term diagnostic utility refers to the percentage of samples that generate any detectable signal. Failure to obtain any detectable signal is most likely due to failure to transfer the cell into the tube or transfer of a cell with degraded DNA (e.g., due to apoptosis). The diagnostic utility of the lymphocyte tests was 99.1%, since only 1 of the 108 reactions did not yield a detectable signal. Diagnostic utility is distinct from diagnostic efficiency, which is the percentage of samples in which the detected signals are strong enough to be scored as robust signals. Only samples that have robust signals should be used to diagnose gender, since weak or delayed signals could be caused by low levels of a contaminant or by suboptimal PCR. In accord with these standards, the two female lymphocyte samples containing weak TSPY signals were scored as undiagnosable. Thus, the overall diagnostic efficiency of this assay as applied to lymphocytes was 98.1% (105/107 samples). Diagnostic accuracy is the percentage of samples correctly scored for gender based on robust signals. Among the 105 samples that displayed only robust signals, all male lymphocytes scored positive for both TSPY and U2, while all female lymphocytes scored positive for U2 only. Therefore, the diagnostic accuracy for this set of lymphocytes was 100%.

### Example 10

### Gene Detection and Gender Diagnosis in Blastomeres

47 non-viable embryos deemed unsuitable for clinical use were analyzed in accordance with Institutional Review Board approvals and patient consent. The embryos were scored according to their level of fragmentation and were disaggregated into individual blastomeres which were tested for TSPY and U2 sequences exactly as described for lymphocytes. The resulting C_{T} and final fluorescence values were used to assess the robustness of the PCR signals based on lymphocyte-generated criteria and to calculate the diagnostic utility, efficiency, and accuracy.

The mean C_{T} and final fluorescence values for TSPY and U2 signals obtained from blastomeres were similar to those of lymphocytes, although the standard deviations were considerably greater for blastomeres (FIG. 4, second experimental series). The higher variability among blastomere measurements is also apparent in FIG. 3. Some of the increased variability was consistent with some blastomeres having completed DNA replication prior to dissection, and therefore having twice the DNA per cell as compared to a quiescent lymphocyte. Doubling the amount of DNA per cell is expected to decrease C_{T} values by about one cycle. Additional causes of increased signal variability for blastomeres are discussed below.

The diagnostic utility for blastomere assays was considerably lower than that of lymphocyte assays. Overall, 185 (74%) of 248 blastomere samples generated at least one signal (FIG. 5). However, when these data are analyzed on the basis of embryo fragmentation, signals were detected in 42 (60%) of 70 blastomeres from highly-fragmented embryos, 46 (74%) of 62 blastomeres from embryos with moderate fragmentation, and 97 (84%) of 116 blastomeres from embryos with low-level fragmentation. The difference in diagnostic utility between blastomeres from embryos with low and high fragmentation was statistically significant (p<0.001) and suggests that embryo quality, rather than experimental technique, was a primary cause for amplification failure. Abnormally developing embryos frequently have anucleate blastomeres, which could account for the absence of signals. In an experiment designed to test this hypothesis, blastomeres with an observed nucleus were assayed for TSPY and/or U2. Signals were detected in 29 (93.5%) of 31 of these blastomeres, significantly different (p<0.01) from the overall rate of 156 (71.9%) of 217 in the remaining blastomeres that were not examined for the presence of a nucleus.

The diagnostic efficiency of blastomere assays was also lower than that of lymphocytes, but did not vary significantly with the degree of embryo fragmentation. Overall, 155 (84%) of 185 blastomere samples with signals exhibited only robust signals (FIGS. 3E-H and FIG. 5). In accord with the criteria defined in Example 9, diagnosis was not made for samples with non-robust signals.

### Example 11

### Diagnostic Accuracy of Blastomere Assays

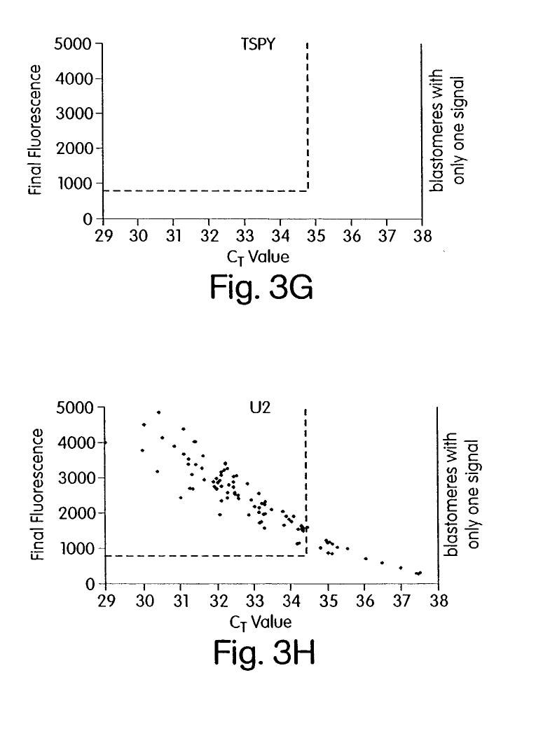

In contrast to lymphocytes, the diagnostic accuracy of single blastomere assays cannot be determined directly because gender is not known in advance. All blastomeres from the same embryo, however, can be expected to have the same chromosomal composition, unless the embryo is chromosomally mosaic. Thus, in order to establish the diagnostic accuracy of the TSPY/U2 assay for sexing embryos, the concordance of gender diagnosis among multiple blastomeres recovered from single embryos was examined. Diagnostic accuracy, like diagnostic utility, improved with embryo quality (FIG. 6). For embryos with high levels of fragmentation, 29 of 33 blastomeres from 11 embryos generated a diagnosis consistent with that obtained from the other blastomeres from the same embryo. Thus, if each blastomere is viewed as a separate test of an embryo, an accurate diagnosis was obtained in 87.9% of those cases. In contrast, all but 1 of 39 blastomeres from embryos with moderate fragmentation were concordant with others from the same embryo, yielding a 97.4% diagnostic accuracy. Consistent with the possibility of non-disjunction, all 5 embryos with non-concordant blastomeres contained only one cell diagnosed as female, while one or more additional cells were diagnosed as male. All 78 blastomeres from embryos with low fragmentation were concordant, yielding a diagnostic accuracy of 100% for that group. The difference in percent concordance between the low and high fragmentation groups was statistically significant (p<0.01).

### Example 12

### Comparison of Various Lysis Conditions

In order for the amplification methods of the invention to be optimized, the lysis conditions of cells must be optimized to maximize the release of DNA from other components of the cell while minimizing cellular damage. Accordingly, a number of different conditions, including commercially available lysis buffers, were compared.

Preparation and Handling of Lymphocytes

Mononuclear leukocytes (mainly lymphocytes) were isolated from the blood of male donors by centrifugation on Histopaque-1077 (Sigma, St. Louis, Mo.). The cells were washed 3 times in PBS, resuspended in 70% PBS, 30% glycerol, and frozen in liquid nitrogen until needed. 1 μl of thawed cell suspension was added to 3 ml PBS in a Costar ultra-low-attachment culture plate (Fisher Scientific, Pittsburgh, Pa.). A single cell was aspirated into a finely-drawn glass pipette while viewing at 100× magnification with an Olympus IX70 microscope. The pipette contents were expelled directly into 10 μl of lysis solution in a 0.2 ml MicroAmp optical PCR tube (PE Applied Biosystems, Foster City, Calif.). The tube was kept on ice until the transfer of all cells was complete.

Protease-Based Lysis

The lysis buffer described in Example 3 was used for the protease-based lysis protocol. Samples of single lymphocytes in lysis buffer were transferred from ice to a preheated thermal cycler block and incubated 30 minutes at 50° C. (or other lysis temperature, as indicated below), and then at 95° C. for 10 minutes.

Freeze-Thaw in Water

The method of Chong, S. S. et al. ((1993) Hum. Molec. Genet. 2:1187-1191) was used with only slight modification. Lymphocytes were transferred to 10 μl of water (18 Megohm, molecular biology grade, Sigma). Samples were initially maintained on ice, slowly frozen to −20° C., then heated to 37° C. Freezing and thawing were repeated for a total of 3 cycles.

Heat Denaturation/Freeze-Thaw in Water

A modification of the method of Schaaff, F. et al. ((1996) Hum. Genet. 98:158161) was used. Lymphocytes were transferred to 10 μl of water. Samples were initially maintained on ice, then placed in a thermal cycler and heated to 95° C. for 10 minutes, cooled, and immediately frozen on dry ice. Samples were thawed at room temperature. Freezing and thawing were repeated for a total of 3 cycles.

Alkaline Lysis

The procedure of Cui et al. as modified by Gitlin, S. A. et al. ((1997) Mol. Hum. Reprod. 3:975-958) was used. Lymphocytes were transferred directly into 5 μl of the published KOH solution (200 mM potassium hydroxide (KOH), 50 mM dithiothreitol (DTT)), or KOH solution with no DTT. The samples were heated at 65° C. for 10 minutes. 5 μl of neutralizing solution (900 mM Tris-HCl, pH 8.3, 300 mM KCl, 200 mM HCl) was then added. In some experiments, KCl was omitted from the neutralizing solution in order to reduce the final concentration of potassium in the PCR reaction to 20 mM.

Commercially Available Lysis Buffers

Single lymphocytes were prepared as described above but were lysed according to the manufacturer's instructions in one of the following commercially available lysis buffers: Microlysis (Microzone Ltd.), Lyse-N-Go (Pierce), Release It (CPG), or Gene Releaser (BioVentures).

PCR Conditions

15 μl of concentrated PCR reagent mixture was added to each tube containing a lysed cell (or a no-cell control) giving final concentrations of 100 mM Tris, pH 8.3, 3.5 mM MgCl₂, 0.4 mM each dNTP, 0.3 μM each primer, 0.3 μM each molecular beacon probe molecule, and 1 unit Taq polymerase (Promega, Madison, Wis.) per 25 μl reaction. Taq polymerase was preincubated with TaqStart antibody (Clontech, Palo Alto, Calif.) for 5 minutes at room temperature before it was added to the PCR mixture to inhibit polymerase activity until the first denaturation step (hotstart PCR). In the tests of alkaline lysis vs. protease-based lysis buffer, all samples were brought to a final volume of 50 μl with the same final concentrations, and potassium concentrations were brought to either 20 or 50 mM.

Amplification and fluorescence detection was carried out using an ABI Prism 7700 Sequence Detector (PE Applied Biosystems). The cycling profile included an initial denaturation step at 95° C. for 3 minutes, followed by 45 cycles of 95° C. for 10 seconds, 58° C. for 45 seconds, and 72° C. for 10 seconds, with fluorescence readings taken during the 58° C. step. For reactions containing KCl, the 72° C. PCR extension step was increased from 10 seconds to 30 seconds to compensate for the inhibitory effect of potassium on Taq polymerase activity. Detection threshold for determining C_{T} values was set at 10 standard deviations above baseline fluorescence readings. Molecular Beacon Probes and Primer Molecules

Primers and molecular beacons were based on the published sequences of the TSPY gene, which is repeated 27 to 40 times on the Y chromosome (Zhang, J. S. et al. (1992) Hum. Mol. Genet. 1:717-726). Molecular beacon probes were designed according to the methods of Tyagi, S. and Kramer, F. R. (1996) as detailed on the internet site: http://molecular-beacons.org and in Pierce et al. (2000). The sense primer sequence was 5′ ATACAGGGCTTCTCATTCCA 3′ (SEQ ID NO: 4) and the antisense primer sequence was 5′ GTTAGATCCTGCGAAGTTGTG 3′ (SEQ ID NO: 5). The TSPY molecular beacon probe sequence was 5′ CGCGCTTTGTGGTGTCTGCGGCGATAGGCAGCGCG 3′ (SEQ ID NO: 6) with the fluorophore TET covalently attached to the 5′ end and the quencher DABCYL covalently attached to the 3′ end.

Statistical Analysis of C_{T} Values

Samples that showed no amplification were regarded as cell transfer failures and were not included in the analysis. Each group of samples was evaluated for the presence of outliers using the Extreme Studentized Deviate (ESD) statistic. Two samples in these experiments with C_{T} values several cycles after the mean for their groups were identified as statistical outliers and were not included in the data analysis of the sample groups. The remaining C_{T} values were evaluated using the F test for the equality of variances. Mean C_{T} values were compared using the t test for independent samples.

Protease-Based Lysis Buffer vs. Water Lysis Methods

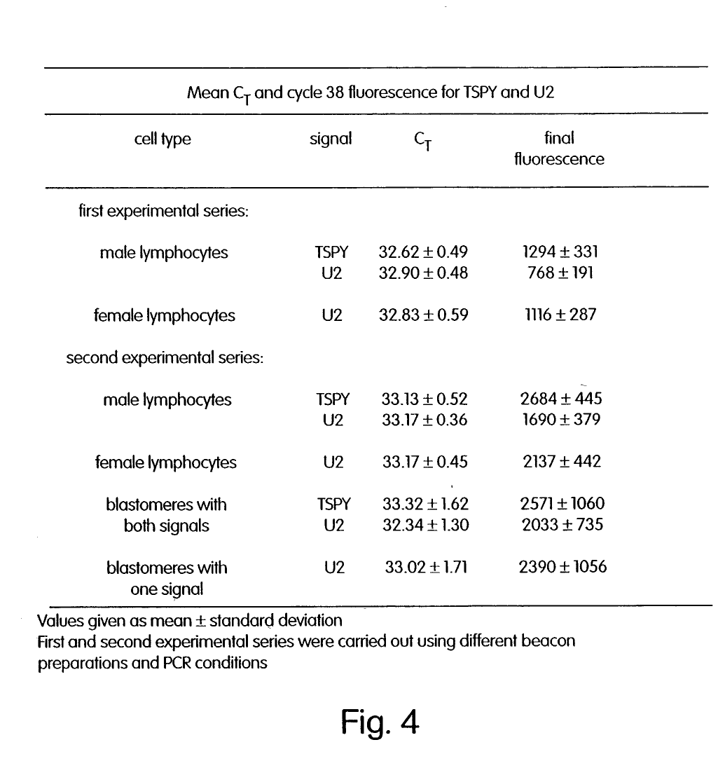

In a comparison of TSPY amplification following lysis of single lymphocytes in protease-based lysis buffer or water, as amplicon accumulates, an increasing number of molecular beacon probe molecules find their homologous targets and assume a conformation that fluoresces (Tyagi and Kramer (1996)). The use of molecular beacon probes provides detection specificity, since only amplicon from the intended targets generates an increase in fluorescence, whereas products from mispriming events are not recognized. An increasing molecular beacon probe fluorescence is seen in the samples measured during each PCR annealing step. The point at which the fluorescence crosses the detection threshold is the C_{T} value of the reaction and is proportional to the number of the approximately 30 TSPY genes that begin amplification during the first few PCR cycles. Once amplification is initiated, PCR efficiency during subsequent cycles has no effect on differences in mean C_{T} values, since PCR conditions are identical for samples within each experiment (unless otherwise indicated). (It should be noted, however, that each of FIGS. 7-12 represents an experiment in which some PCR variables may differ from those of previous experiments, including the use of different molecular beacon probe preparations, so accurate comparisons cannot be made using C_{T} values from these figures.)

PCR results clearly demonstrate that availability of DNA for amplification following incubation in protease-based lysis buffer is vastly better than it is following repeated freeze-thaw in water. The C_{T} value for each sample is shown in FIG. 7. The difference between the mean C_{T} value of 34.39 for the protease-based lysis buffer samples and the mean C_{T} values of 39.41 for freeze-thaw in water is highly significant (p<0.001). FIG. 7 also shows the C_{T} values for samples that were heat denatured in water prior to freeze-thaw. Including the 95° C. incubation step prior to freeze-thaw provided improved results for water lysis, with a mean C_{T} value of 35.00, suggesting that early denaturation of endogenous nucleases or chromatin proteins is important for this technique. However, detection was significantly later than in the protease-based lysis buffer samples (p<0.01).

Alkaline Lysis and the Effect of DTT

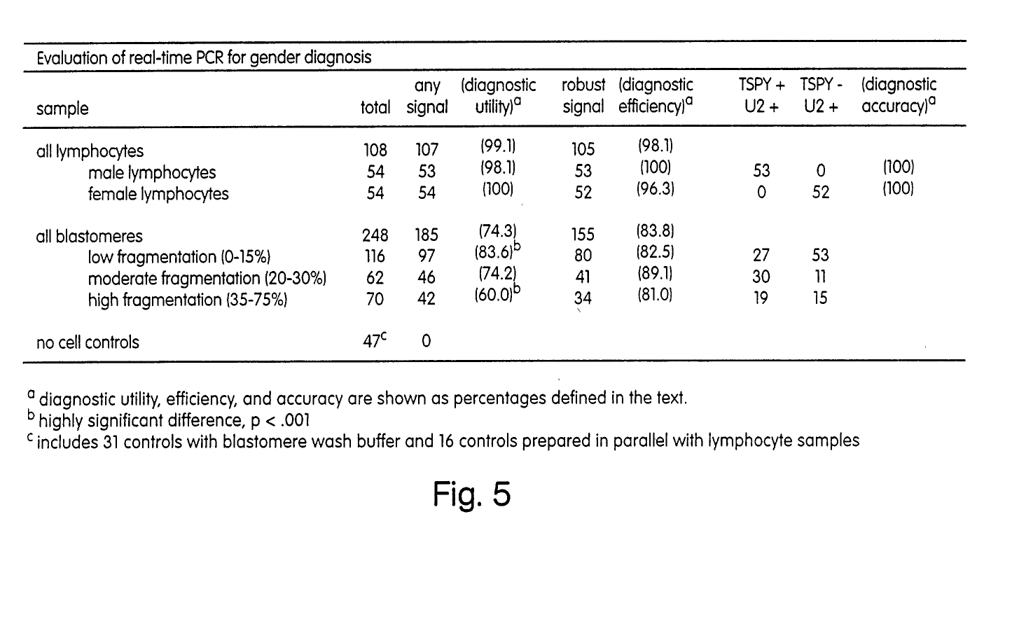

Initial tests of the alkaline lysis technique yielded C_{T} values that averaged 2 to 3 cycles later than those obtained with protease-based lysis buffer. In order to test the possibility that this large difference might be due to the effects of residual DTT on amplification efficiency rather than DNA target availability, comparisons were made of alkaline lysis containing 50 mM DTT (standard protocol), 5 mM DTT, or no DTT (FIG. 8). Lowering the DTT concentration to 5 mM resulted in a significant reduction in the mean C_{T} value from 36.16 to 35.40 (p<0.05). Eliminating DTT from the lysis solution further reduced the mean C_{T} value to 33.76, a highly significant difference from each of the other groups (p<0.001).

Protease-Based Lysis Buffer vs. Alkaline Lysis Without DTT

In order to accurately compare the protease-based lysis buffer and alkaline lysis protocols, some adjustments were made in the PCR conditions. Since the standard alkaline lysis protocol provides a concentration of 50 mM potassium for the PCR reaction, KCl was added to the PCR mix for protease-based lysis buffer samples. It has been observed that potassium ions have variable effects on PCR efficiency depending on the specific amplicon. Including 20 or 50 mM potassium was found to lower the C_{T} values for TSPY, although the highest concentration resulted in an increased variance among replicate samples. Therefore, lysis protocols were also tested using a final concentration of 20 mM potassium. This was possible with the alkaline lysis protocol by eliminating the KCl included in the standard neutralization buffer.

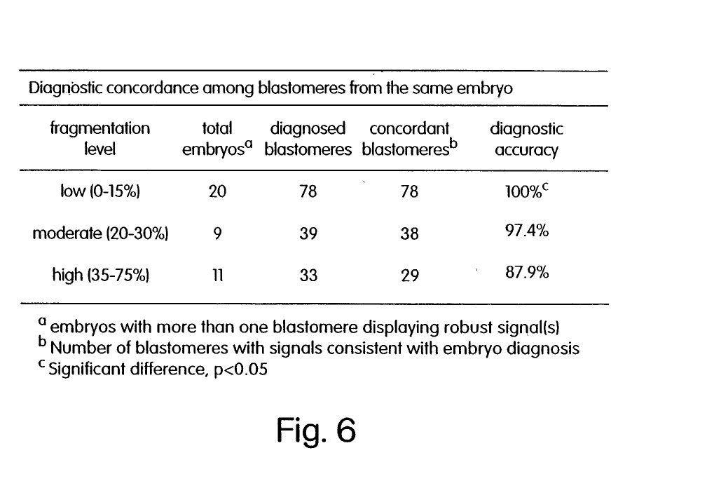

FIG. 9 presents the comparison of protease-based lysis buffer and alkaline lysis (without DTT) using a final concentration of 20 mM potassium for PCR. Similar results were obtained using 50 mM potassium. The two lysis protocols yielded equivalent mean C_{T} values. The small difference in variance was not significant.

Substitution of Nonionic Detergents for SDS in the Protease-Based Lysis Buffer

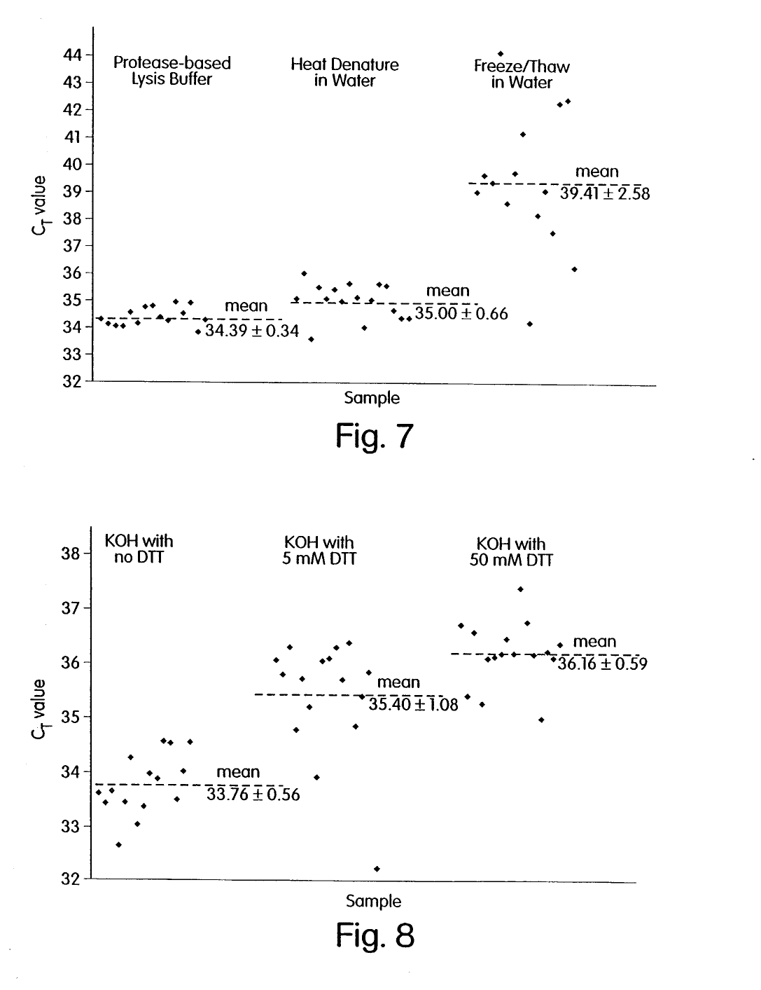

Some published lysis protocols utilize nonionic detergents such as Triton X-100 or Nonidet P-40 (NP-40). Comparisons of samples treated with protease-based lysis buffer containing either Triton X-100 or NP-40 are shown in FIG. 10. The mean C_{T} values of 34.99 obtained using Triton X-100 are 35.25 obtained using NP-40 were significantly higher than the mean C_{T} value of 34.10 obtained with protease-based lysis buffer containing SDS (p<0.001).

Effect of the Presence of Magnesium on Lysis Using Proteinase K

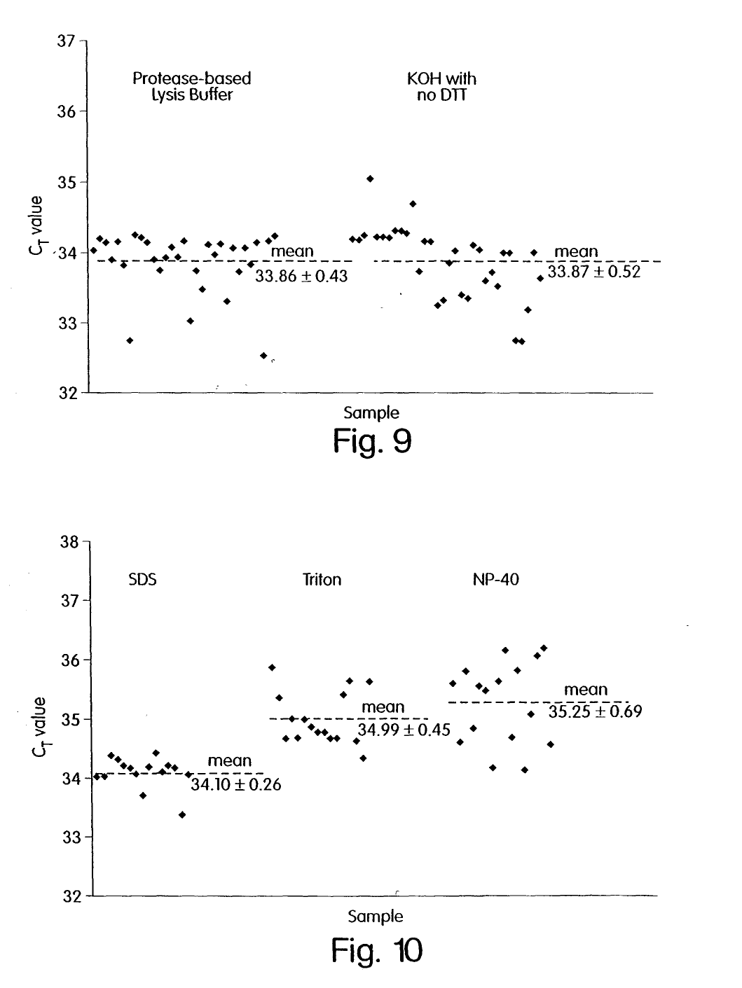

Magnesium is added to lysis buffers by some investigators, possibly because it can increase the half-life of proteinase K activity and because it is necessary in subsequent PCR reactions. FIG. 11 compares samples incubated in protease-based lysis buffer containing proteinase K with samples incubated in protease-based lysis buffer containing proteinase K and 1.5 mM MgCl₂. The presence of magnesium increased the mean C_{T} value from 33.01 to 34.15. The increase was statistically significant (p<0.01). Higher concentrations of magnesium were found to cause even greater increases in C_{T} values, although the increase could be partially negated through the use of higher incubation temperatures. These results suggest that a reduction in DNA target availability may be the result of reduced proteinase K activity.

Protease-Based Lysis Buffer vs. Commercially Available Lysis Buffers

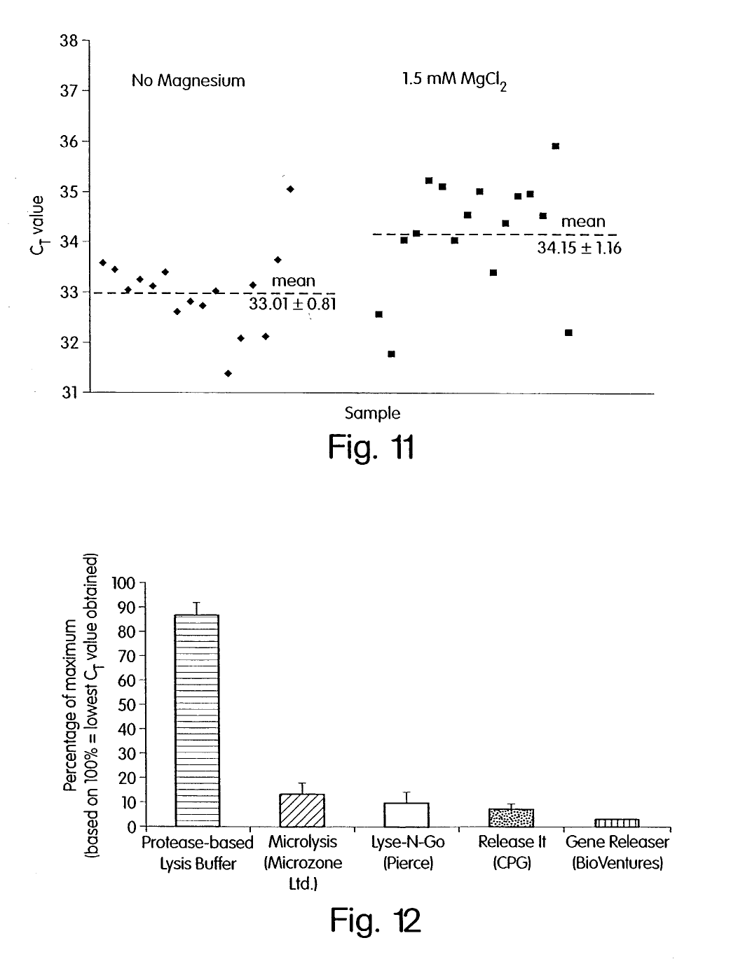

As shown in FIG. 12, use of the protease-based lysis buffer yielded far greater amplification of TSPY targets than did use of any of the commercially available lysis buffers for amplification of targets from single cells.

### Example 13

### Cell Lysis and PCR in a Single Tube Containing All Required Reagents

A lysis/amplification mixture was prepared which contained primers and molecular beacon probes specific for TSPY and U2 (all at 300 nM), 10 mm dNTPs, 5 μM SDS, 100 μg/ml proteinase K, and 100 mM Tris, pH 8.3. This mixture was pipetted in 50 μl aliquots to PCR tubes. One wax bead containing about 1.25 units of Taq polymerase and one wax bead containing sufficient MgCl₂ to provide a final concentration of 3 mM were added to each tube. Single male lymphocytes were transferred to the solution in 8 tubes, and single female lymphocytes were transferred to the solution in 8 other tubes. 6 μg of purified male DNA (equivalent to 1 genome) was added to each of 4 tubes containing the same buffer for positive controls, and 4 negative control tubes received no added cells or DNA.

Fluorescence readings were taken before (background) and after the lysis/amplification reactions for each molecular beacon probe fluor (FAM for male, HEX for female) and were measured with a Bio-Tek FL600 fluorescence reader. These measurements (taken through the bottom of the PCR tube) were needed since fluorescence detection using an ABI 7700 is done through the cap and is partially obscured by the presence of the melted wax. Lysis and amplification reactions were done in the ABI 7700 using the following program: 50° C. for 30 minutes, 95° C. for 10 minutes, and then 45 cycles of 95° C. for 30 seconds, 58° C. for 30 seconds, and 72° C. for 30 seconds. Fluorescence was acquired during the 58° C. step of each cycle. The presence of specific amplicons was confirmed by electrophoresis of the PCR sample through a 3% agarose gel.

The male-specific TSPY signal (FAM) increase was detected in 7 of 8 male lymphocyte samples using the ABI 7700. The mean C_{T} value of 36.3 (with a range of 36.0 to 36.6) is 2 to 3 cycles higher than previously observed for lysed male lymphocytes, but this is likely due to interference of signal detection by the wax. Positive TSPY signal (relative to control) was detected using the BioTek reader for those same 7 male samples. Gel electrophoresis confirmed amplicon of the expected size for those samples.

U2 (control) signal (HEX) increase was detected in 7 of 8 female lymphocyte samples using the ABI 7700, although the increase was only slightly over background levels in some samples. Positive signal (relative to control) was detected in 6 of those samples. The relatively weak sample from the HEX fluorophore may be responsible for the relatively poor signal detection. None of the female samples showed TSPY signal increase. Gel electrophoresis confirmed a single amplicon of the expected size for U2 in 7 samples.

Equivalents

Those skilled in the art will recognize, or will be able to ascertain using no more than routine experimentation, many equivalents to the specific embodiments of the invention described herein. Such equivalents are intended to be encompassed by the following claims.

What is claimed is:

1. An isolated moderately-repeated highly-conserved nucleic acid sequence that is:
a) repeated 3-100 times within the genome of a cell or part thereof; and
b) sufficiently conserved such that at least two non-overlapping oligonucleotide primer molecules are able under stringent conditions to hybridize to and permit the amplification of the plurality of the copies of said nucleic acid sequence.

2. An oligonucleotide primer, comprising a nucleotide sequence that is sufficiently complementary to the moderately-repeated highly-conserved nucleic acid sequence of claim 1 to permit hybridization of the primer under stringent conditions to a plurality of the copies of said moderately-repeated highly-conserved nucleic acid sequence present in the nucleic acid molecules comprising the genomes of fewer than 10 cells or part thereof.

3. The primer of claim 2, wherein said primer is complementary to the moderately-repeated highly-conserved nucleic acid sequence only at the 5′ and 3′ ends of the primer molecule, such that hybridization of the primer to said moderately-repeated highly-conserved sequence results in the circularization of the primer molecule.

4. The primer of claim 2, wherein the nucleic acid molecules are isolated from a mammalian cell.

5. The primer of claim 4, wherein the mammalian cell is a human cell.

6. The primer of claim 5, wherein said primer is sufficiently complementary to a moderately-repeated highly-conserved nucleic acid sequence contained within human chromosome 17 to permit hybridization under stringent conditions to a plurality of the copies of said moderately-repeated highly-conserved nucleic acid sequence in human chromosome 17.

7. The primer of claim 6, wherein said primer comprises the nucleotide sequence of SEQ ID NO: 1.

8. The primer of claim 6, wherein said primer comprises the nucleotide sequence of SEQ ID NO: 2.

9. The primer of claim 5, wherein said primer is sufficiently complementary to a moderately-repeated highly-conserved nucleic acid sequence contained within the human Y chromosome to permit hybridization under stringent conditions to a plurality of the copies of said moderately-repeated highly-conserved nucleic acid sequence in the human Y chromosome.

10. The primer of claim 9, wherein said primer comprises the nucleotide sequence of SEQ ID NO: 4.

11. The primer of claim 9, wherein said primer comprises the nucleotide sequence of SEQ ID NO: 5.

12. The primer of claim 2, wherein said primer includes a detectable label.

13. The primer of claim 12, wherein the label is detectable only when said primer is hybridized to the moderately-repeated highly-conserved nucleic acid sequence or its specific amplicon.

14. The primer of claim 12, wherein the label is detectable only when said primer is not hybridized to the moderately-repeated highly-conserved nucleic acid sequence or its specific amplicon.

15. An oligonucleotide probe, comprising a nucleotide sequence that is sufficiently complementary to the moderately-repeated highly-conserved nucleic acid sequence of claim 1 to permit hybridization of the primer under stringent conditions to a plurality of the copies of said moderately-repeated highly-conserved nucleic acid sequence present in the nucleic acid molecules comprising the genomes of fewer than 10 cells or part thereof.

16. The probe of claim 15, wherein the nucleic acid molecules are isolated from a mammalian cell.

17. The probe of claim 16, wherein the mammalian cell is a human cell.

18. The probe of claim 17, wherein said probe is sufficiently complementary to a moderately-repeated highly-conserved nucleic acid sequence contained within human chromosome 17 to permit hybridization under stringent conditions to a plurality of the copies of said moderately-repeated highly-conserved nucleic acid sequence in human chromosome 17.

19. The probe of claim 18, wherein said probe comprises the nucleotide sequence of SEQ ID NO: 3.

20. The probe of claim 17, wherein said probe is sufficiently complementary to a moderately-repeated highly-conserved nucleic acid sequence contained within the human Y chromosome to permit hybridization under stringent conditions to a plurality of the copies of said moderately-repeated highly-conserved nucleic acid sequence in the human Y chromosome.

21. The probe of claim 20, wherein said probe comprises the nucleotide sequence of SEQ ID NO: 6.

22. The probe of claim 15, wherein said probe includes a detectable label.

23. The probe of claim 22, wherein: the detectable label comprises a fluor and a quencher such that in the absence of hybridization of said probe to the moderately-repeated highly-conserved nucleic acid sequence or its specific amplicon, said probe forms a hairpin loop structure that brings said fluor and quencher sufficiently proximate such that fluorescence is substantially quenched; and wherein upon hybridization of said probe to said moderately-repeated highly-conserved nucleic acid sequence or its specific amplicon, said fluor and quencher are separated and a fluorescent signal is emitted.

24. The probe of claim 22, wherein the label is detectable only when the probe is not hybridized to the moderately-repeated highly-conserved nucleic acid sequence or its specific amplicon.

25. A method of detecting the presence or quantity of a nucleic acid sequence present in a sample of nucleic acid molecules comprising the genomes of fewer than 10 cells or part thereof, comprising:
a) contacting the cells or part thereof with a protease-based lysis buffer comprising:
i) an ionic detergent;
ii) a protease; and
iii) a buffering agent, to form a mixture;
b) incubating the mixture at a temperature at which the protease is active such that a sample of substantially accessible nucleic acid molecules is obtained;
c) incubating the sample at a temperature at which the protease is substantially inactivated;
d) contacting the sample with at least one nucleic acid primer complementary to a plurality of the copies of said nucleic acid sequence;
e) amplifying said nucleic acid sequence by an amplification reaction; and
f) detecting the amplicon of the nucleic acid sequence as indicative of the presence or quantity of said nucleic acid sequence in said sample.

26. The method of claim 25, wherein the nucleic acid sequence is a moderately-repeated highly-conserved nucleic acid sequence.

27. The method of claim 26, wherein the moderately-repeated highly-conserved sequence is contained within human chromosome 17.

28. The method of claim 27, wherein the amplification reaction is a cyclical amplification reaction and the sample is contacted with two primers comprising the nucleotide sequences of SEQ ID NO: 1 and SEQ ID NO: 2, respectively.

29. The method of claim 26, wherein the moderately-repeated highly-conserved sequence is contained within the human Y chromosome.

30. The method of claim 29, wherein the amplification reaction is a cyclical amplification reaction and the sample is contacted with two primers comprising the nucleotide sequences of SEQ ID NO: 4 and SEQ ID NO: 5, respectively.

31. The method of claim 25, wherein the sample is contacted with two primers complementary to opposite strands of the nucleic acid sequence and wherein the amplification reaction is a cyclical amplification reaction.

32. The method of claim 25, wherein the sample is contacted with at least two primer molecules comprising:
a) a first primer having both a 3′ and 5′ end complementary to nonoverlapping regions of the nucleic acid sequence such that upon hybridization, the primer forms a circular structure containing a gap; and
b) at least a second primer which is complementary to a sequence found between the 3′ and 5′ ends of the first primer molecule, such that the second primer is able to hybridize to the first primer and fill the gap between the ends of the first primer; and
wherein the amplification reaction is a rolling circle amplification reaction.

33. The method of claim 25, wherein at least one of the primers includes a detectable label.

34. The method of claim 33, wherein the label is detectable only when the primer is hybridized to the nucleic acid sequence or its specific amplicon.

35. The method of claim 33, wherein the label is detectable only when the primer is not hybridized to the nucleic acid sequence or its specific amplicon.

36. The method of claims 25, wherein the step of detecting comprises:
a) contacting the sample with an oligonucleotide probe which specifically hybridizes to a plurality of the copies of the nucleic acid sequence or its specific amplicon; and
b) detecting said probe.

37. The method of claim 36, wherein the probe includes a detectable label.

38. The method of claim 37, wherein: the detectable label comprises a fluor and a quencher such that in the absence of hybridization of said probe to the nucleic acid sequence or its specific amplicon, said probe forms a hairpin loop structure that brings said fluor and quencher sufficiently proximate such that fluorescence is substantially quenched; and wherein upon hybridization of said probe to said nucleic acid sequence or its specific amplicon, said fluor and quencher are separated and a fluorescent signal is emitted.

39. The method of claim 37, wherein the label is detectable only when the probe is not hybridized to the nucleic acid sequence or its specific amplicon.

40. The method of claim 25, wherein the amplicon is detected or quantified in real time during the amplification reaction.

41. The method of claim 25, wherein at least one of the primers includes a detectable label and the amplicon is detected or quantified in real time during the amplification reaction by measuring the label associated with the primer hybridized to the amplicon.

42. The method of claim 36, wherein the probe contains a detectable label and the amplicon is detected or quantified in real time during the amplification reaction by measuring the label associated with the probe hybridized to the amplicon.

43. The method of claim 42, further comprising the step of comparing the quantity of amplicon detectable at a first selected time of amplification and the quantity of amplicon detectable at a later second selected time of amplification to predetermined quantity values for the first and second selected times of amplification as an indication of the presence or quantity of the amplification reaction.

44. The method of claim 42, further comprising the step of comparing the quantity of amplicon detectable at a first selected time of amplification and the quantity of amplicon detectable at a later second selected time of amplification to predetermined quantity values for the first and second selected times of amplification as an indication of the efficiency of the amplification reaction

45. The method of claim 25, wherein the sample of nucleic acid molecules is isolated from a single cell.

46. The method of claim 25, wherein the sample of nucleic acid molecules is isolated from one part of one cell.

47. The method of claim 25, wherein the protease is proteinase K.

48. The method of claim 25, wherein the temperature at which the protease is active is about 50° C.

49. The method of claim 25, wherein the temperature at which the protease is substantially inactivated is about 95° C.

50. The method of claim 25, wherein the buffering agent maintains the pH of the reaction at or near the optimal pH for the activity of the protease.

51. The method of claims 25, wherein the buffering agent is Tris.

52. The method of claim 25, wherein the buffering agent maintains the pH above 7.2 at the incubation temperature of the first incubation step.

53. The method of claim 25, wherein the ionic detergent is sodium dodecyl sulfate.

54. The method of claim 25, wherein the lysis buffer does not include chaotropic salts or Mg²⁺.

55. The method of claim 25, wherein the first incubation step lasts about one hour.

56. The method of claim 25, wherein the protease is proteinase K; the buffering agent is Tris; the ionic detergent is sodium dodecyl sulfate; and the lysis buffer does not include chaotropic salts or Mg²⁺.

57. The method of claim 25, wherein:
a) the entirety of the method is conducted in a sealed reaction vessel; and
b) polymerase and Mg²⁺ molecules are added to the sample in a form such that they are made available for the amplification step only after the step wherein the protease is inactivated.

58. The method of claim 57, wherein the polymerase and Mg²⁺ molecules are encased in wax, and wherein the wax is melted and the polymerase and Mg²⁺ molecules are made available for the amplification step during the step wherein the protease is inactivated.

59. The method of claim 25, wherein the protease-based lysis buffer is replaced by an alkaline lysis buffer that does not contain DTT or any other reducing agent; wherein the first incubation step lasts for a time sufficient to obtain substantially accessible nucleic acid molecules; and wherein, prior to the amplification, the pH of the sample is neutralized by the addition of an acid and a buffering agent.

60. The method of claim 59, wherein the alkaline lysis buffer contains potassium hydroxide.

61. A method for preparing a sample of accessible nucleic acid molecules from fewer than 10 cells or parts thereof for an amplification reaction comprising:
a) contacting the cells or part thereof with an alkaline lysis buffer that does not contain DTT or any other reducing agent, to form a mixture;
b) incubating the mixture for an amount of time sufficient to obtain substantially accessible nucleic acid molecules;
c) neutralizing the pH mixture by adding an acid and a buffering agent,
such that substantially accessible nucleic acid molecules are obtained.

62. The method of claim 61, wherein the alkaline lysis buffer contains potassium hydroxide.

63. A method for preparing a sample of substantially accessible nucleic acid molecules from fewer than 10 cells or parts thereof for an amplification reaction, comprising:
a) contacting the cells or parts thereof with a protease-based lysis buffer comprising:
i) an ionic detergent
ii) a protease; and
iii) a buffering agent,
to form a mixture;
b) incubating the mixture at a temperature at which the protease is active;
such that substantially accessible nucleic acid molecules are obtained.

64. The method of claim 63, wherein the protease is proteinase K.

65. The method of claim 63, wherein the temperature is about 50° C.

66. The method of claim 63, wherein the buffering agent maintains the pH of the reaction at or near the optimal pH for the activity of the protease.

67. The method of claim 63, wherein the buffering agent is Tris.

68. The method of claim 63, wherein the buffering agent maintains the pH above 7.2 at the incubation temperature.

69. The method of claim 63, wherein the ionic detergent is sodium dodecyl sulfate.

70. The method of claim 63, wherein the lysis buffer does not include chaotropic salts or Mg²⁺.

71. The method of claim 63, wherein the step of incubating lasts about one hour.

72. The method of claim 63, further comprising inactivating the protease prior to the amplification step.

73. The method of claim 63, wherein the protease is proteinase K; the buffering agent is about Tris; the ionic detergent is sodium dodecyl sulfate; and the lysis buffer does not include chaotropic salts or Mg²⁺.

74. A protease-based lysis buffer comprising:
a) an ionic detergent;
b) a protease; and
c) a buffering agent sufficient to achieve and maintain a pH of about 7.2 or above at the incubation temperature of a method in which the lysis buffer is utilized, and wherein chaotropic salts and Mg²⁺ are not included in the lysis buffer.

75. The protease-based lysis buffer of claim 74, wherein the ionic detergent is sodium dodecyl sulfate, the protease is proteinase K, and the buffering agent is Tris.

76. A kit for the preparation of substantially accessible nucleic acid molecules from the nucleic acid molecules comprising the genomes of fewer than 10 cells or part thereof comprising: a protease-based lysis buffer comprising an ionic detergent, a protease, and a buffering agent; in at least a first container.

77. The kit of claim 76, wherein the lysis buffer does not include chaotropic salts or Mg²⁺.

78. The kit of claim 76, wherein the protease is proteinase K, the ionic detergent is sodium dodecyl sulfate, and the buffering agent is Tris.

79. A kit for detecting the presence or quantity of a nucleic acid sequence present in a sample of nucleic acid molecules comprising the genomes of fewer than 10 cells or part thereof, comprising:
a) a protease-based lysis buffer comprising an ionic detergent, a protease, and a buffering agent, in at least a first container; and
b) at least one oligonucleotide primer which specifically hybridizes to a plurality of the copies of said nucleic acid sequence, in at least a second container.

80. The kit of claim 79, wherein the nucleic acid sequence is a moderately-repeated highly-conserved nucleic acid sequence.

81. The kit of claim 80, wherein the moderately-repeated highly-conserved nucleic acid sequence is contained in the human Y chromosome.

82. The kit of claim 80, wherein the moderately-repeated highly-conserved nucleic acid sequence is contained in human chromosome 17.

83. The kit of claim 79, wherein at least one of the primers includes a detectable label.

84. The kit of claim 83, wherein the label is detectable only when the primer is hybridized to the nucleic acid sequence or its specific amplicon.

85. The kit of claim 83, wherein the label is detectable only when the primer is not hybridized to the nucleic acid sequence.

86. The kit of claim 79, further comprising at least a third container containing an oligonucleotide probe which specifically hybridizes to a plurality of the copies of the nucleic acid sequence or its specific amplicon.

87. The kit of claim 86, wherein the probe includes a detectable label.

88. The kit of claim 87, wherein: the detectable label comprises a fluor and a quencher such that in the absence of hybridization of said probe to the nucleic acid sequence or its specific amplicon, said probe forms a hairpin loop structure that brings said fluor and quencher sufficiently proximate such that fluorescence is substantially quenched; and wherein upon hybridization of said probe to said nucleic acid sequence or its specific amplicon, said fluor and quencher are separated and a fluorescent signal is emitted.

89. The kit of claim 87, wherein the label is detectable only when the probe is not hybridized to the nucleic acid sequence or its specific amplicon.

90. The kit of claim 79, further comprising an amplification reagent comprising:
a) a polymerase;
b) a buffering agent;
c) one or more salts; and
d) deoxynucleotide triphosphate molecules.

91. The kit of claim 90, wherein the polymerase is provided encased in wax.

92. The kit of claim 91, further comprising Mg²⁺ molecules encased in wax.

93. The kit of claim 79, further comprising at least one enhancer of a molecular beacon probe.

94. The kit of claim 79, further comprising a plurality of primers which specifically hybridize to two or more nucleic acid sequences or their specific amplicons.

95. The kit of claim 79, further comprising a plurality of probes which specifically hybridize to two or more nucleic acid sequences or their specific amplicons.

96. The kit of claim 79, further comprising a plurality of enhancers of molecular beacon probes.

97. A method of preparing gene-deleted DNA for use in an amplification reaction comprising contacting a sample of complete-genome DNA with a sequence specific replication inhibitor, such that at least the first replication event of a specific nucleic acid sequence in the amplification reaction is prevented or delayed.

98. A sample of gene-deleted DNA prepared by the method of claim 97.

99. The method of claim 97, wherein the sequence specific replication inhibitor is a protein having an enzymatic activity.

100. The method of claim 97, where in the sequence specific replication inhibitor is an oligonucleotide.

101. The method of claim 97, wherein the sequence specific replication inhibitor is a non-enzymatic protein.

102. The method of claim 97, wherein the sequence specific replication inhibitor is at least one small molecule.

103. A method for selecting best-possible primers, comprising:
a) performing the methods of either of claims 25 or 59, wherein at least one pair of primers is used to contact the sample; and
b) determining whether the primers are best-possible primers, wherein best possible primers are those that, when used in the method herein, have at least one of the following properties:
i) lower C_{T} value;
ii) smaller C_{T} value variance;
iii) higher fluorescence 4-6 cycles beyond the C_{T} value;
iv) smaller variance of the fluorescence 4-6 cycles beyond the C_{T} value;
v) a greater rate of signal increase; and
vi) fewer non-specific amplicons
than other primer pairs specific for the same nucleic acid sequence.

104. A method for selecting best-possible primers, comprising:
a) performing the methods of either of claims 25 or 59, wherein:
i) at least one pair of primers is utilized to contact the sample;
ii) the sample of nucleic acid molecules comprising the genomes of fewer than 10 cells or part thereof is replaced by a nucleic acid sample comprising the genomes 10 or more cells or part thereof; and
iii) wherein the nucleic acid sample is derived from gene-deleted DNA; and
b) determining whether the primers are best-possible primers, wherein best possible primers are those that, when used in the method herein, result in the fewest non-specific amplicons, as compared to other primers specific for the same nucleic acid sequence.

105. An enhancer of a probe, wherein said enhancer keeps an amplicon in a single-stranded or unhybridized state in the region where said probe hybridizes to its target sequence.

106. The enhancer of claim 105, wherein said enhancer is an oligonucleotide.

107. The enhancer of claim 105, wherein said enhancer is a protein having an enzymatic activity.

108. The enhancer of claim 105, wherein said enhancer is a non-enzymatic protein.

109. The enhancer of claim 105, wherein the probe is a molecular beacon.


SEQUENCE LISTING (8 sequences)

SEQ ID NO: 1 - DNA, 19 bp, Artificial Sequence
  Description of Artificial Sequence primer
aagaaatcagcccgagagt

SEQ ID NO: 2 - DNA, 20 bp, Artificial Sequence
  Description of Artificial Sequence primer
cttgatcttagccaaaaggt

SEQ ID NO: 3 - DNA, 35 bp, Artificial Sequence
  Description of Artificial Sequence primer
ctggcctgtctcgtccacagcgctattgaggccag

SEQ ID NO: 4 - DNA, 20 bp, Artificial Sequence
  Description of Artificial Sequence primer
atacagggcttctcattcca

SEQ ID NO: 5 - DNA, 21 bp, Artificial Sequence
  Description of Artificial Sequence primer
gttagatcctgcgaagttgtg

SEQ ID NO: 6 - DNA, 35 bp, Artificial Sequence
  Description of Artificial Sequence primer
cgcgctttgtggtgtctgcggcgataggcagcgcg

SEQ ID NO: 7 - DNA, 20 bp, Artificial Sequence
  Description of Artificial Sequence primer
aaaggcaacgtccaggatag

SEQ ID NO: 8 - DNA, 20 bp, Artificial Sequence
  Description of Artificial Sequence primer
aattcttcggcagcatcttc